(19) United States
(12) Patent Application Publication
(10) Pub. No.: US 2025/0108372 A1
(43) Pub. Date: Apr. 3, 2025

(54) MICROFLUIDIC SYSTEMS CONTAINING LAYERS OF FILMS

(71) Applicant: President and Fellows of Harvard College, Cambridge, MA (US)

(72) Inventors: Mohamed Yafia Okba Salem, Cambridge, MA (US); Adama Marie Sesay, Cambridge, MA (US); Pawan Jolly, Cambridge, MA (US); Donald E. Ingber, Cambridge, MA (US)

(73) Assignee: President and Fellows of Harvard College, Cambridge, MA (US)

(21) Appl. No.: 18/832,918

(22) PCT Filed: Jan. 24, 2023
(86) PCT No.: PCT/US2023/011472

§ 371 Date: Jul. 24, 2024

Related U.S. Application Data
(60) Provisional application No. 63/302,832, filed on Jan. 25, 2022.
(60) Provisional application No. 63/302,863, filed on Jan. 25, 2022.
(60) Provisional application No. 63/302,865, filed on Jan. 25, 2022.

(51) Int. Cl. B01L 3/00

(52) U.S. Cl. CPC B01L 3/502715; B01L 2200/12; B01L 2200/16; B01L 2300/0883; B01L 2300/161; B01L 2400/0487; B01L 2400/0622

(57) ABSTRACT

Systems and methods related to microfluidic devices (e.g., microfluidic devices comprising layers of films) are generally described. In some embodiments, a microfluidic device comprises a substrate configured to facilitate fluid transport, one or more intermediate layers disposed on the substrate, wherein the one or more intermediate layers are configured to define a plurality of fluidly connected microfluidic components, and a top layer disposed on the one or more intermediate layers. In certain embodiments, a microfluidic device comprises a microfluidic channel having a gap or area of increased hydrophobicity in between two separated portions of the channel to separately pin one or more liquids in one or more desired portions of the channel. According to some embodiments, a microfluidic device comprises a microfluidic channel with an inclined surface, such that different portions of the microfluidic channel are associated with different channel heights.

### RELATED APPLICATIONS

This application claims priority under 35 U.S.C. § 119 (e) to U.S. Provisional Patent Application No. 63/302,832, filed Jan. 25, 2022, U.S. Provisional Patent Application No. 63/302,863, filed Jan. 25, 2022, and U.S. Provisional Patent Application No. 63/302,865, filed Jan. 25, 2022, each of which are incorporated herein by reference in their entirety for all purposes.

### TECHNICAL FIELD

Systems and methods related to microfluidic devices (e.g., microfluidic devices comprising layers of films) are generally described.

### BACKGROUND

Passive microfluidic platforms, such as lateral flow assays (LFAs), are commonly used for point of care diagnostics due to their low cost and simplistic microfluidic capabilities. LFAs, however, have limited microfluidic capabilities and cannot be used for performing advanced microfluidic operations (e.g., mixing and diluting liquids, delivering different liquid samples and/or reagents sequentially, stopping liquid flow for incubation and restarting the flow for sensing, etc.). For this reason, active microfluidic systems with multiple bulky pumps, actuators, controllers, mechanical valves, and/or power sources are required to perform these advanced microfluidic operations. Capillary microfluidic systems offer advantages that LFAs do not provide, since the aforementioned advanced microfluidic operations can be accomplished using capillary action. Conventional microfluidic devices with capillary functions require specifically designed geometries and are therefore produced by advanced fabrication techniques using expensive equipment.

### SUMMARY

Systems and methods related to microfluidic devices (e.g., microfluidic devices comprising layers of films) are generally described. The subject matter of the present disclosure involves, in some cases, interrelated products, alternative solutions to a particular problem, and/or a plurality of different uses of one or more systems and/or articles.

According to some embodiments, a microfluidic device is described, the microfluidic device comprising a substrate configured to facilitate fluid transport; one or more intermediate layers disposed on the substrate, wherein the one or more intermediate layers are configured to define a plurality of fluidly connected microfluidic components; and a top layer disposed on the one or more intermediate layers.

In certain embodiments, a method of manufacturing a microfluidic device is described, the method comprising applying one or more intermediate layers to a substrate to define a plurality of fluidly connected microfluidic components; and applying a top layer to a topmost one of the one or more intermediate layers.

According to certain embodiments, a microfluidic device is described, the microfluidic device comprising a plurality of microfluidic channels, wherein at least a portion of at least one microfluidic channel has a transverse cross-section that is orthogonal to a direction of flow through the at least one microfluidic channel, and wherein a first portion of the transverse cross-section has a first hydrophobicity or hydrophilicity and a second portion of the transverse cross-section has a second hydrophobicity or hydrophilicity that is different than the first hydrophobicity or hydrophilicity.

According to some embodiments, a microfluidic device is described, the microfluidic device comprising a substrate configured to facilitate fluid transport; one or more intermediate layers disposed on the substrate, wherein the one or more intermediate layers are configured to at least partially define a plurality of microfluidic channels; and a top layer disposed on the one or more intermediate layers, wherein a bottom surface of at least a portion of at least one microfluidic channel has a first hydrophilicity or hydrophobicity and one or more side walls of at least a portion of the at least one microfluidic channel has a second hydrophilicity or hydrophobicity that is greater than the first hydrophilicity or hydrophobicity.

In certain embodiments, a microfluidic device is described, the microfluidic device comprising a substrate configured to facilitate fluid transport; an intermediate layer disposed on the substrate, wherein the intermediate layer is configured to at least partially define a first microfluidic channel and a second microfluidic that intersect with each other; and a top layer disposed on the one or more intermediate layers, wherein an interface between the first microfluidic channel and the second microfluidic channel is patterned such that the interface is configured to function as a stop valve.

According to some embodiments, a microfluidic device is described, the microfluidic device comprising a substrate configured to facilitate fluid transport; one or more intermediate layers disposed on the substrate, wherein the one or more intermediate layers are configured to at least partially define a microfluidic channel; and a hydrophobic layer disposed on the one or more intermediate layers, wherein the hydrophobic layer is more hydrophobic than the intermediate layers, and wherein a portion of the hydrophobic layer exposed to an interior of a portion of the microfluidic channel has a first transverse dimension at an upstream location and a second transverse dimension at a downstream location, wherein the first transverse dimension is smaller than the second transverse dimension such that the portion of the microfluidic channel is configured to act as a retention valve.

Some embodiments are related to a microfluidic device comprising a channel including a first portion extending along a length of the channel; a second portion extending along the length of the channel; and a third portion extending along the length of the channel wherein the third portion is disposed between the first portion of the channel and the second portion of the channel, and wherein the third portion is configured to isolate the first portion of the channel from the second portion of the channel until a liquid is flowed through the third portion of the channel.

Some embodiments are related to a microfluidic device comprising a substrate; a first intermediate film layer disposed on the substrate, wherein a recessed channel is formed in the first intermediate film layer; and a second intermediate film layer disposed on the first intermediate film layer, wherein a primary channel is formed in the second intermediate film layer, and wherein a first portion of the primary channel is disposed on a first side of the recessed channel and a second portion of the primary channel is disposed on a second side of the recessed channel opposite from the first side.

Some embodiments are related to a method of operating a microfluidic device. In certain embodiments, the method comprises flowing a first liquid through a recessed channel disposed between a first portion of a primary channel and a second portion of a primary channel; and mixing a first substance in the first portion of the primary channel with a second substance in the second portion of the primary channel after the fluid is flowed through the recessed channel.

In some embodiments, a microfluidic device comprises a first layer, a second layer disposed on the first layer, and a third layer disposed on to the second layer, wherein a portion of the third layer is directly disposed on the first layer, and wherein the portion of the third layer forms at least a portion of a microfluidic channel.

In some embodiments, a microfluidic device comprises a microfluidic channel extending in a first direction, the microfluidic channel comprising a first portion and a second portion, wherein the second portion is spaced from the first portion in a second direction transverse to the first direction, wherein a surface of the microfluidic channel includes an inclined portion between the first and second portions, wherein a height of the first portion is less than a height of the second portion, and wherein the heights of the first and second portions extend in a third direction perpendicular to both the first and second directions.

In some embodiments, a method comprises introducing fluid into a first portion of a microfluidic channel, the microfluidic channel extending in a first direction; flowing the fluid in a second direction transverse to the first direction from the first portion of the microfluidic channel to a second portion of the microfluidic channel, wherein the first portion of the microfluidic channel and the second portion of the microfluidic channel are at least partially coextensive along a length of the microfluidic channel; and flowing the fluid out of the second portion of the microfluidic channel to an outlet of the microfluidic channel, the outlet spaced from the first portion in the first direction.

Other advantages and novel features of the present disclosure will become apparent from the following detailed description of various non-limiting embodiments when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand. In the figures:

FIG. 1A shows, according to some embodiments, a cross-sectional schematic diagram of a microfluidic device comprising a substrate, an intermediate layer, and a top layer;

FIG. 1B shows, according to some embodiments, a cross-sectional schematic diagram of a microfluidic device comprising a substrate, a first intermediate layer, a second intermediate layer, and a top layer;

FIG. 1C shows, according to some embodiments, a cross-sectional schematic diagram of a microfluidic device comprising a substrate, a first intermediate layer, a second intermediate layer, a third intermediate layer, and a top layer;

FIGS. 2A-2D show, according to some embodiments, a cross-sectional schematic diagram depicting a method of fabricating a microfluidic device;

FIG. 3A shows, according to some embodiments, a top-view schematic diagram of a microfluidic device;

FIG. 3B shows, according to some embodiments, a cross-sectional schematic diagram along line 3B in FIG. 3A of a first intermediate layer, a second intermediate layer, and a third intermediate layer defining a microfluidic channel;

FIG. 3C shows, according to some embodiments, a cross-sectional schematic diagram along line 3C in FIG. 3A of a first intermediate layer, a second intermediate layer, and a third intermediate layer defining a valve;

FIG. 4A shows, according to some embodiments, a top-view schematic diagram of a substrate;

FIG. 4B shows, according to some embodiments, a top-view schematic diagram of a first intermediate layer;

FIG. 4C shows, according to some embodiments, a top-view schematic diagram of a second intermediate layer;

FIG. 4D shows, according to some embodiments, a top-view schematic diagram of a third intermediate layer;

FIG. 4E shows, according to some embodiments, a top-view schematic diagram of a top layer;

FIG. 4F shows, according to some embodiments, a top-view schematic diagram of a microfluidic device containing the layers shown in FIGS. 4A-4E in a stacked configuration;

FIG. 5A-5C show, according to some embodiments, top-view schematic diagrams of portion 5A-5C in FIG. 3A;

FIG. 6A shows, according to some embodiments, a first intermediate layer defining at least one microfluidic channel and reservoir;

FIG. 6B shows, according to some embodiments, a second intermediate layer defining at least one microfluidic channel and reservoir;

FIG. 7A shows, according to some embodiments, a top-view schematic diagram of another top layer;

FIG. 7B shows, according to some embodiments, a top-view schematic diagram of another third intermediate layer;

FIG. 7C shows, according to some embodiments, a top-view schematic diagram of another second intermediate layer;

FIG. 7D shows, according to some embodiments, a top-view schematic diagram of another first intermediate layer;

FIG. 7E shows, according to some embodiments, a top-view schematic diagram of another substrate;

FIG. 8 shows, according to some embodiments, a cross-sectional schematic diagram of a microfluidic channel having three portions;

FIG. 9A shows, according to some embodiments, a cross-sectional schematic diagram of a microfluidic device;

FIG. 9B shows, according to some embodiments, the cross-sectional schematic diagram of the microfluidic device in FIG. 9A with pinned fluids in portions of the channel;

FIG. 10A shows, according to some embodiments, a portion of a microfluidic channel;

FIG. 10B shows, according to some embodiments, a cross-section of the microfluidic channel of FIG. 10A;

FIG. 11A shows, according to some embodiments, a portion of a microfluidic channel including a reagent;

FIG. 11B shows, according to some embodiments, a cross-section of the microfluidic channel of FIG. 11A;

FIG. 12A shows, according to some embodiments, a microfluidic channel including an inlet and an outlet;

FIG. 12B shows, according to some embodiments, a cross-section of the microfluidic channel of FIG. 12A;

FIG. 13A shows, according to some embodiments, another portion of a microfluidic channel;

FIG. 13B shows, according to some embodiments, a cross-section of the microfluidic channel of FIG. 13A;

FIG. 14A shows, according to some embodiments, yet another portion of a microfluidic channel;

FIG. 14B shows, according to some embodiments, a cross-section of the microfluidic channel of FIG. 14A

FIG. 15 shows, according to some embodiments, a microfluidic device comprising an electrochemical detection sensor;

FIG. 16 shows, according to some embodiments, current in response to the detection of samples containing COVID-19 antibodies compared to a control;

FIGS. 17A-17B show embodiments illustrating the effect of surface treatment on hydrophilicity or hydrophobicity, in accordance with some embodiments;

FIG. 17C-17D show embodiments illustrating the effect of different surfaces of films on hydrophilicity or hydrophobicity, in accordance with some embodiments;

FIG. 18A shows, according to some embodiments, a schematic diagram of a microfluidic device comprising a channel having a middle portion disposed between a first portion and a second portion;

FIG. 18B shows, according to some embodiments, a schematic diagram of the microfluidic device of FIG. 18A with a first substance in the first portion of the channel and a second substance in the second portion of the channel;

FIG. 19A shows, according to some embodiments, an embodiment of the schematic diagram shown in FIG. 18A; and

FIG. 19B shows, according to some embodiments, an embodiment of the schematic diagram shown in FIG. 18B.

### DETAILED DESCRIPTION

Systems and methods related to microfluidic devices (e.g., microfluidic devices comprising layers of films) are generally described.

Passive capillary action-driven microfluidic systems offer advantages that lateral flow assays (LFAs) and active microfluidic systems do not provide, because advanced microfluidic operations can be accomplished using surface tension forces associated with capillary action to drive multiple functions. A variety of passive capillary elements may be used to perform these advanced microfluidic operations successfully. Conventional capillary microfluidic devices without active components require specifically designed geometries and/or additional surface treatments to create multiple functional capillary elements. These passive capillary elements perform different functions during loading and running the microfluidic device, which increases their design complexity to be able to perform the required advanced operations without using active elements. For example, the passive capillary elements may have small feature sizes (e.g., 10-1,500 microns), and they need to be designed with certain surface properties and specific geometries to be able to control the capillary pressure precisely. Microfluidic devices with advanced functionalities based on capillary action therefore require specifically designed geometries, and such devices are produced by advanced fabrication techniques that require expensive equipment.

Furthermore, certain fabrication techniques that are currently employed use materials that need additional surface treatment steps (e.g., plasma treatment, salinization) to tailor contact angles and control the capillary pressure at specific locations within the microfluidic device. This surface treatment, however, must be stable when long storage times are needed during product development. Moreover, current conventional surface treatment procedures cannot control the surface treatment location on the microfluidic device, as all surfaces of the device are exposed evenly to the surface treatment modifications.

To this end, the Inventors have recognized and appreciated that there is a desire for lower cost and technically easier methods for fabricating microfluidic devices driven by passive capillary action while maintaining the ability to perform advanced microfluidic operations by imparting different functionalities to different portions of the microfluidic device.

Described herein are new designs, materials, and methods for fabrication of passive capillary elements that can precisely modify and control the capillary pressure at different sections of a microfluidic device. The Inventors have recognized and appreciated that the capillary pressure can be controlled by: (i) the contact angle on hydrophilic and hydrophobic surfaces; and (ii) the geometry of the passive capillary elements. According to some embodiments, these passive elements may perform advanced microfluidic operations such as sequential liquid delivery steps, hydrating dried reagents, and mixing and diluting liquids, with stable surface properties and without costly surface modification techniques.

According to some embodiments, microfluidic devices that comprise one or more patterned films where each film has at least a portion of a profile of a desired portion of the microfluidic device formed in the one or more patterned films are described herein. The one or more patterned films are stacked together (e.g., bonded) to form the microfluidic device. In certain embodiments, for example, the microfluidic device comprises: a substrate; one or more patterned intermediate layers that define a plurality of passive elements (e.g., channels, valves, reservoirs, other microfluidic components); and a top layer to cover the microfluidic device. In some embodiments, the substrate is configured to facilitate fluid flow through one or more microfluidic channels of the microfluidic device. The one or more intermediate layers, in some embodiments, each comprise a film (e.g., a polymer film) that may have either the same or varying degrees of hydrophobicity and/or hydrophilicity. In either case, a top layer may be applied over the last intermediate layer. The microfluidic devices described herein advantageously comprise functional passive elements configured to perform advanced capillary microfluidic operations without using active pumps, valves, or power sources.

The disclosed microfluidic devices may be fabricated by applying the one or more intermediate layers over the substrate such that the first intermediate layer binds (e.g., adheres) to the substrate and/or another intermediate layer. Depending on the embodiment, the one or more intermediate layers may include an adhesive material applied to one or both opposing sides of the film layers to bond each layer to an adjacent layer and/or the substrate. For example, in some embodiments, a pressure sensitive adhesive may be used. However, embodiments in which the films of a microfluidic device are assembled without the use of adhesives are also contemplated. For instance, in some embodiments, the film layers may be bonded to one another using appropriate lamination techniques and/or any other appropriate bonding method as the disclosure is not so limited. Specific bonding techniques are described in further detail below.

The use of individual layers to form the different portions of the microfluidic components may enable the use of different materials for various portions of a microfluidic device, which facilitates the fabrication of devices with tailored surface properties. For instance, the hydrophobicity and/or hydrophilicity of the different layers may vary from layer to layer, or the hydrophobicity and/or hydrophilicity may vary within the same layer. Thus, a device may be configured such that different variations and combinations of hydrophobic and hydrophilic materials may be used to form the substrate, the one or more intermediate layers, and/or the top layer of a microfluidic device. In addition, the thickness of one or more layers, their geometries, and/or their dimensions may be tailored such that the one or more layers contribute to controlling capillary pressures throughout the device. Such a configuration advantageously enables the fabrication of microfluidic devices that can be fine-tuned to perform advanced microfluidic operations, including sequential delivery and/or mixing of liquid samples and/or reagents, timed and/or stepped operations using sacrificial reservoirs that can act as timers, stopping and incubating liquid samples, and starting and stopping flow through a device using sacrificial reservoirs, by precisely controlling the capillary pressures at desired locations within the device. Furthermore, the fabrication methods described herein that provide the ability to fine-tune the hydrophilicity and/or hydrophobicity of the one or more layers of the device may eliminate the need for additional surface treatments, resulting in devices with longer shelf lives and less expensive production costs.

As described above, the microfluidic devices described herein may comprise a plurality of connected microfluidic components, such as channels, valves, and/or reservoirs. In some embodiments, for example, one or more layers of the microfluidic device may define one or more microfluidic channels. One or more properties (e.g., surface properties, physical properties) of at least a portion of a microfluidic channel may be tailored such that the portion of the microfluidic channel is configured to act as a valve (e.g., a stop valve, a retention valve). As explained in greater detail herein, for example, the hydrophobicity or hydrophilicity of a portion of a channel may be modified relative to other components of or the remainder of the channel, such that the modified portion is configured to act as a stop valve. In other embodiments, the geometry of a portion of channel may be constructed such that the portion of the channel is configured to act as a retention valve.

Stop valves are configured to stop the flow of liquid at specific positions within the microfluidic device. Conventional techniques used to fabricate stop valves involve surface treatment methods in which the channel surfaces are rendered hydrophilic or hydrophobic. Low surface tension liquids in such conventional microfluidic devices, however, may leak from upper channels to lower channels resulting in leakage through the stop valve. Described herein are two-stepped and single-layer capillary stop valves formed from hydrophilic and hydrophobic materials that advantageously enable the ability to work with low surface tension liquid samples and reagents.

According to some embodiments, a microfluidic channel may be defined by more than one layer of the microfluidic device (e.g., a substrate and/or one or more intermediate layers, one or more intermediate layers) such that the hydrophobicity varies within the same channel. In some embodiments, for example, a microfluidic channel may have a transverse cross-section that is orthogonal to a direction of flow through the microfluidic channel, wherein a first portion of the transverse cross-section has a first hydrophobicity and a second portion of the transverse cross-section has a second hydrophobicity that is different from the first hydrophobicity. Configuring the microfluidic channel in this way advantageously provides a stop valve in which a fluid may flow along a comparatively less hydrophobic portion of the channel and stop at the comparatively more hydrophobic portion of the channel.

In certain embodiments, a microfluidic channel may be defined by more than one layer of the device (e.g., a substrate and/or one or more intermediate layers, one or more intermediate layers) such that the surfaces and/or side walls of the microfluidic channel vary in hydrophobicity relative to one or more adjacent layers. In some embodiments, for example, the bottom surface of a microfluidic channel may be defined by a substrate or a first intermediate layer, and may be rendered hydrophilic, and the side walls of the microfluidic channel may be defined by one or more intermediate layers, and may be rendered hydrophobic or less hydrophilic than the bottom surface. In some such embodiments, the configuration of the microfluidic device advantageously allows the side walls of the microfluidic channel to stop low surface tension liquids from leaking and breaking the stop valve.

According to some embodiments, the surface properties of a single intermediate layer may be selectively modified to fabricate a stop valve. For example, a portion of a microfluidic channel defined by an intermediate layer may be patterned to change the degree of hydrophilicity or hydrophobicity of the surface of the portion relative to an adjacent portion of the microfluidic channel and provide spatial wettability inside the channel. The single layer stop valve can advantageously be used to stop the flow in one channel and trigger flow from another channel on the same intermediate layer, which can be done by defining patterns (e.g., by laser etching and/or engraving) in the microfluidic channel to enable the merging of two liquids effectively at intersecting channels. As explained in greater detail herein, various patterns, such as a triangular pattern, may be used to stop and restart the flow and merge liquids.

Other mechanisms of controlling the capillary pressure in the microfluidic channels, besides tuning the hydrophobicity and/or hydrophilicity of the channels, include changing the geometry and/or dimensions of the channel to facilitate capillary action. In some embodiments, for example, the geometry of a hydrophobic or hydrophilic portion of a microfluidic channel may be configured such that the hydrophobic or hydrophilic portion of the channel acts as a valve (e.g., a stop valve, a retention valve). A portion of the microfluidic channel may be fluidly connected to a liquid reservoir, and the hydrophobic portion of the microfluidic channel may include a first geometry with a first transverse dimension (e.g., a width perpendicular to a longitudinal axis of the channel, a depth perpendicular to a longitudinal axis of the channel) that transitions into a second narrower geometry with a second smaller transverse dimension. The change in the width and/or depth of the microfluidic channel may permit the portion of the microfluidic channel to act as a retention valve due to the increased capillary pressure at the section of the microfluidic channel with the smaller transverse dimension. One way in which to provide such a construction is a hydrophobic layer with a cut out with a narrower upstream geometry and a wider downstream geometry as elaborated on further below. In one specific embodiment, a suitable geometry may include, for example, a triangular geometry, wherein the narrow portion of the triangular geometry is upstream, proximate to a liquid reservoir, and the wide portion of the triangular geometry is downstream.

Some portions and/or sections of the microfluidic device have dual functions when operating the device. An inlet reservoir, for example, should enable facile filling during loading the device and may act as a retention valve to pin the liquid during running the device. In some embodiments, the geometry at one or more inlet reservoirs may enable liquid flow when filling the device, while a comparatively more narrow section of the reservoir inlet acts as a retention valve when running the device.

Some embodiments described herein are related to a microfluidic channel having a gap or area of increased hydrophobicity in between two separated portions of the channel to separately pin one or more liquids in one or more desired portions of the channel. In some embodiments, for example, a channel has a middle portion disposed between two opposing portions of the channel extending along a length of the channel. The middle portion may isolate liquids separately contained in one or both of the opposing portions of the channel, which may also be referred to as the two flanking portions of the channel. The isolation may be provided by a capillary stop valve effect, for example by making at least one surface of the middle portion more hydrophobic than adjacent surfaces of the two flanking portions. A liquid may be added in the middle portion such that liquids in the two flanking portions are intermixed by forming one single liquid block inside the channel. The intermixing may be used to perform intermixing of different liquids separately isolated on the flanking portions, or be used to reconstitute one or more dried reagents held in one or both flanking portions of the channel.

In some embodiments, the middle portion of a channel is a recessed portion disposed on a substrate or an intermediate layer, and having two side walls adjacent to the respective flanking portions of the channel. Isolation may be formed by making at least one of the bottom surfaces or side walls of the recessed portion more hydrophobic than adjacent surfaces of the flanking portions of the channel.

In some embodiments, the channel having a gap or area of increased hydrophobicity in between two separated portions of the channel may be constructed from patterned layers of hydrophilic and hydrophobic films and polymers. These layers are designed to precisely modify and control the capillary pressure at different sections for fabricating various functional capillary elements without using active pumps, valves, and/or power sources. This method enables fabrication of structures with different surface properties, providing precise control over capillary pressures at desired locations. The structures also can have different surface properties on the same layer by modifying the hydrophilicity or hydrophobicity of the surface in defined patterns to provide spatial wettability. The top and/or the bottom surface of the layers may, in some embodiments, be treated with a hydrophobic or hydrophilic coating to control the capillary pressure. The thickness of the layers and their geometries may contribute to controlling the capillary pressure and varying the capillary pressure at different locations. Different fabrication and cutting methods of the layers can affect the surface properties on the side walls of the layers, which may result, for example, in increased differences in hydrophobic properties of different portions of the channel. Accordingly, the capillary pressure can be also tuned by choosing the fabrication and cutting method. For example, stronger isolation between portions of the channel may be created due to increased differences in hydrophobicity. Combining layers with different surface properties and patterned surface treatment enables fabrication of capillary structures with more robust functionalities to work with virtually all liquids, samples, and reagents, even those with low surface tensions.

In certain embodiments, a microfluidic channel may be defined by more than one layer of the microfluidic device (e.g., a substrate and/or one or more intermediate layers, one or more intermediate layers) such that the channel geometry (e.g., channel height, channel inclination angle) varies within the same channel. In some embodiments, for example, a microfluidic channel may have a transverse cross-section that is orthogonal to a direction of flow through the microfluidic channel, wherein a first portion of the transverse cross-section has a first channel height, and a second portion of the transverse cross-section has a second channel height that is different from the first channel height. Configuring the microfluidic channel in this way may advantageously provide a capillary pressure gradient in which a fluid may flow from a portion of the channel with comparatively high capillary pressure toward a portion of the channel with comparatively low capillary pressure. Accordingly, the geometry of a microfluidic channel may be designed to encourage fluid flow in a direction transverse to a length of the microfluidic channel.

According to some embodiments, for example, described herein are microfluidic devices that comprise microfluidic channels with inclined surfaces, such that different portions of the microfluidic channel are associated with different channel heights. By tailoring the height of the microfluidic channel in different portions of the channel, different capillary pressure in different portions of the channel may be provided. Accordingly, certain flow properties of a fluid within the microfluidic channel may be controlled by designing the geometry of the microfluidic channel appropriately. A microfluidic channel with an inclined surface may enable a smooth, continuous transition between different portions of the microfluidic channel with different channel heights.

In some embodiments, an inclined surface of a microfluidic channel may be inclined in a direction that is aligned with the direction in which the microfluidic channel extends. For example, at least a portion of a microfluidic channel may extend between an inlet and an outlet of the channel. The inlet may be associated with a shorter channel height than the outlet. Accordingly, the microfluidic channel may be inclined in the direction in which the microfluidic channel extends (e.g., in a direction from the inlet to the outlet) such that a fluid in the channel flows down the inclined surface from the inlet to the outlet. A capillary pressure gradient from the inlet to the outlet may urge fluid introduced in the inlet to flow toward the outlet.

In some embodiments, an inclined surface of a microfluidic channel may be inclined in a direction that is transverse to the direction in which the microfluidic channel extends. A microfluidic channel with a transverse inclination may promote flow in a direction transverse to the length of the microfluidic channel. For example, if a microfluidic channel extends from an inlet to an outlet, a first portion of the microfluidic channel (e.g., the left side) may extend along at least a portion of a length of the channel and may be associated with a shorter channel height than a second portion of the channel disposed on and extending along at least a portion of an opposite side of the microfluidic channel (e.g., the right side). Accordingly, a capillary pressure gradient from the first portion of the microfluidic channel to the second portion of the microfluidic channel may urge fluid to flow from the first portion of the microfluidic channel to the second portion of the microfluidic channel. It should be appreciated that additional parameters (e.g., other channel dimensions, fluid surface tension) may affect the flow patterns and/or flow directions within a particular microfluidic channel. In some embodiments, the first portion of the microfluidic channel may fill before any transverse flow (e.g., from the first portion toward the second portion) occurs. In some embodiments, transverse flow may begin before the first portion fills completely. In some embodiments, the second portion of the microfluidic channel may fill before the first portion of the microfluidic channel fills as a fluid is introduced into an inlet of the microfluidic channel.

Such transverse fluid flow may be advantageous in applications that include mixing a fluid with one or more reagents (e.g., dried reagents). In a conventional microfluidic channel with a uniform channel height, a dehydrated reagent disposed within the channel may simply be flushed through the outlet of the channel before sufficient mixing has occurred, as the primary fluid flow is in a single direction from the inlet of the microfluidic channel to the outlet of the microfluidic channel. However, in a microfluidic channel with different heights and one or more inclined surfaces, a dried reagent may be disposed along a length of a microfluidic channel on the side of the channel with an increased channel height (e.g., at the bottom of an inclined surface). The associated transverse flow of fluid within the channel before the fluid exits an outlet of the channel may promote better mixing and more uniform concentrations of the reagent within the fluid along the length of the channel.

In some embodiments, a microfluidic device may include a microfluidic channel with one or more inclined surfaces extending in different directions. For example, a microchannel may include an inclined surface that is inclined at least partly in a direction in which the microfluidic channel extends and that is inclined at least partly in a direction that is transverse to the direction in which the microfluidic channel extends. It should be appreciated that a microfluidic channel may be inclined in any direction or any combination of directions, as the present disclosure is not limited in this regard.

Additionally, it should be appreciated that the disclosure is not limited to layer- or film-based manufacturing methods. For example, a microfluidic channel with an inclined surface may be manufactured using a mold formed from an additive manufacturing process or any other appropriate microfluidic device manufacturing technique as the current disclosure is not limited to how the microfluidic devices are manufactured.

As explained in greater detail, the microfluidic devices described herein may be used for a wide variety of applications, including, for example, electrochemical detection (e.g., affinity-based electrochemical detection). In some embodiments, the microfluidic device may comprise or otherwise be associated with an electrochemical detection sensor. Such devices may be configured to detect viral antibodies, such as COVID-19 antibodies, although other types of antibodies are also possible as the disclosure is not meant to be limiting in this regard.

The disclosed microfluidic devices and associated manufacturing methods offer several benefits relative to conventional microfluidic devices. For example, in some embodiments, the disclosed devices and methods may offer: significant cost reductions; reduced (or no) need for additional surface treatments; longer shelf life due to the use of more stable materials; reduced fabrication and assembly times; the ability to mass-produce microfluidic devices; reducing or eliminating the need for external peripherals and actuators such as valves and pumps; as well as other potential benefits. Combining layers with various surface properties and patterned surface treatment enables fabrication of new capillary structures with robust functionalities that work with a wide array of liquids, samples, and reagents, even those with low surface tensions. Embodiments in which a particular microfluidic device offers benefits different than those noted above are also contemplated as the disclosure is not limited to any particular application and/or design of a microfluidic device.

It should be understood that while specific microfluidic devices with various components such as the illustrated channels, valves, and reservoirs are described relative to the figures, other configurations of microfluidic devices using the methods and systems described herein are also contemplated. Accordingly, it should be understood that the microfluidic devices disclosed herein may comprise any of a variety of additional elements. In some embodiments, for example, a microfluidic pump may be provided to facilitate fluid flow through one or more channels of the microfluidic device. In other embodiments, an electrochemical detection sensor may be coupled to the microfluidic device to facilitate affinity-based electrochemical detection, as explained in greater detail below.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

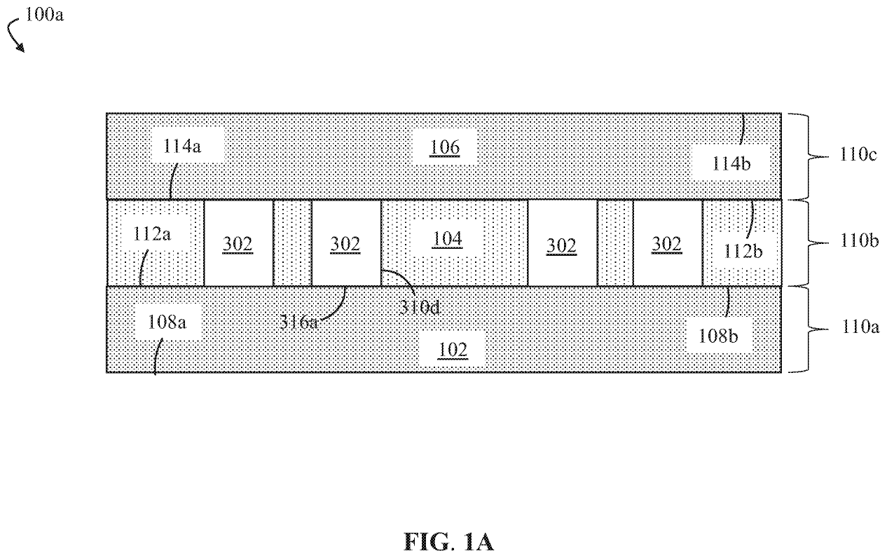

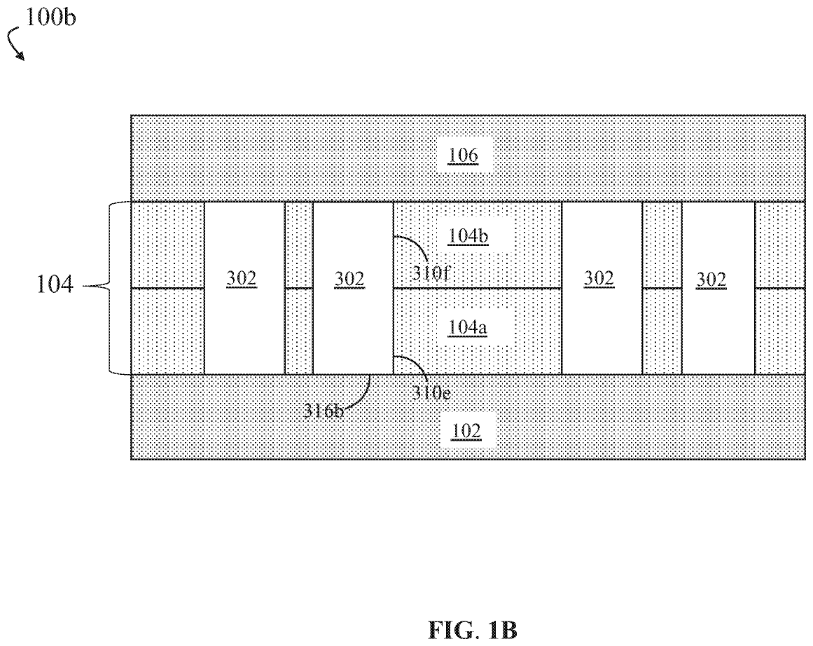

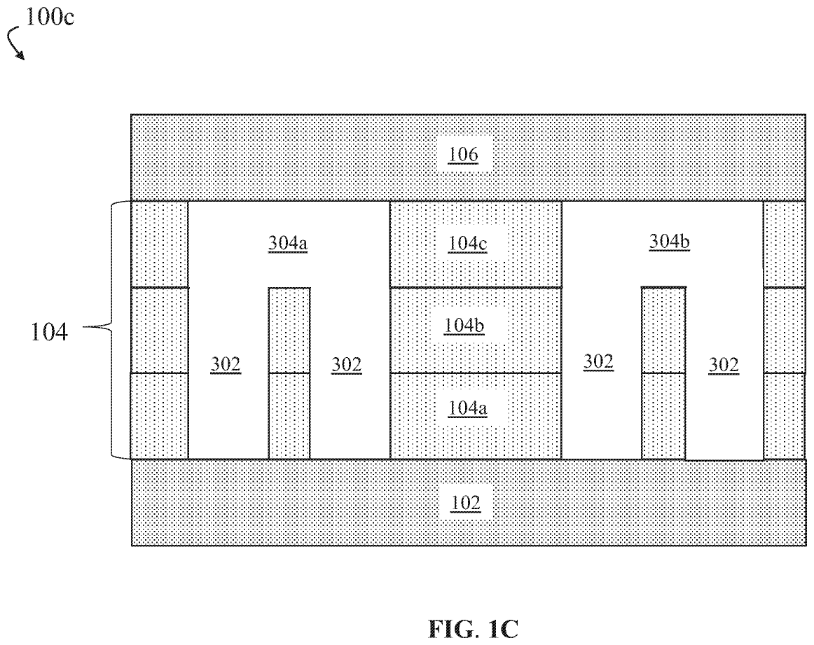

FIG. 1A shows, according to some embodiments, a cross-sectional schematic diagram of microfluidic device 100a comprising substrate 102, at least one intermediate layer 104 disposed on substrate 102, and top layer 106 disposed on at least one intermediate layer 104 opposite substrate 102. Thus, at least one intermediate layer 104 is disposed between substrate 102 and top layer 106, and top layer 106 and substrate 102 may cover the opposing surfaces of one or more intermediate layers 104, which may be patterned to define one or more channels, valves, reservoirs, or other microfluidic components in the microfluidic device. For instance, the one or more intermediate layers are, in some embodiments, patterned to define a plurality of fluidly connected channels, valves, reservoirs, and/or other microfluidic components that are formed when the layers are stacked together. These components may either be hydraulic and/or pneumatic components (e.g., liquid channels and air channels) depending on the particular application. One example of such a system is shown in FIG. 1A where plurality of channels 302 are formed (e.g., cut) in one or more intermediate layers 104. Specifically, portions of intermediate layer 104 have been removed, to form a desired pattern in intermediate layer 104 configured to define the one or more microfluidic components. The microfluidic components are defined by the sides of the removed portions of intermediate layer 104 and the adjacent surfaces of the layers disposed on either side of the intermediate layer. For example, substrate 102 and top layer 106 form the upper and lower surfaces, respectively, of channels 302 in the embodiment depicted in FIG. 1A.

To maintain the various layers of a microfluidic device in a desired configuration, it may be desirable to bond, or otherwise hold, the layers in the desired configuration. For example, adhesives, heat bondable materials, compression, dry lamination techniques, and/or any other appropriate method of holding a stack of layers in a desired configuration may be used. In one such embodiment, an adhesive (e.g., a pressure sensitive adhesive, a photo-curable adhesive, or any other appropriate adhesive) may be used to bond the individual layers to one another. Referring, for example, to FIG. 1A, an adhesive may be disposed between surface 114a of top layer 106 and adjacent surface 112b of underlying intermediate layer 104. Similarly, an adhesive may be present between surface 112a of a bottom most intermediate layer (e.g., intermediate layer 104) and adjacent surface 108b of substrate 102. In some embodiments, an adhesive may present between adjacent surfaces of intermediate layers. When an adhesive material is present between the various layers, it may be applied to either surface prior to bonding as the disclosure is not limited in this fashion. Additionally, in some embodiments, an adhesive may also be applied to an external surface of the stacked layers including, for example, external surface 114b of top layer 106 opposite the one or more intermediate layers and/or external surface 108a of substrate 102 opposite the one or more intermediate layers. The use of an adhesive applied to an external surface of the microfluidic device may allow the substrate and/or top layer to be attached to another object.

As noted above, in some embodiments, an adhesive may be used to bond one or more, and in some instances, all of the layers of a microfluidic device together. In some embodiments using adhesives, the adhesive may be applied as a coating on one or both sides of an individual layer, on a surface of an adjacent layer, and/or as a separate layer disposed between two opposing layers. Additionally, in some embodiments, one or more layers, such as an intermediate layer of a device, may be formed from an adhesive material. Thus, it should be understood that the current disclosure is not limited to where or how an adhesive is applied to bond the top layer, the one or more intermediate layers, and/or the substrate to one another. Appropriate types of adhesives may include, but are not limited to, pressure sensitive adhesives (PSAs), light curable adhesives, delayed-tack adhesives, and/or any other appropriate type of adhesive as the disclosure is not so limited. For example, in certain embodiments, a layer may include a film made from a PSA (e.g., a silicone PSA), a polymer, or a copolymer film coated with a PSA (e.g., a polyester, polypropylene, or styrene-ethylene-butylene-styrene film coated with a PSA). Specific examples may include, but are not limited to, a single-sided silicone transfer adhesive with two polyester film release liners (e.g., ARclad® IS-8026); a double-sided polypropylene adhesive coated on both sides with a silicone pressure sensitive adhesive between two polyethylene terephthalate (PET) release liners (e.g., ARseal™ 90880); a single-sided hydrophobic microfluidic diagnostic tape with delayed-tack adhesive (e.g., 9795R from 3M™), and/or a single-sided hydrophilic pressure-sensitive adhesive protected by a clear siliconized polyester release liner (e.g., ARflow® 93049 (Developmental)).

In addition to the above, it is also possible that at least two of the individual layers of a microfluidic device may be bonded to each other without the use of adhesives. For example, in some embodiments, one or more layers of a microfluidic device may either be laminated to one another using a heat-based process, a chemical bonding process of the layers (e.g., dry bond lamination), and/or any other appropriate non-adhesive based bonding method, such as, for example, plasma activated bonding and/or extrusion lamination. Alternatively, the individual layers may be mechanically held in a desired configuration using clamps, fixtures, or other mechanical arrangements that apply a compressive force to the stacked layers to maintain the layers in a desired configuration during operation.

While specific embodiments using adhesives and non-adhesive bonding are described above, it should be understood that the disclosure is not limited to how the individual layers are bonded to one another. Accordingly, a stack of individual layers may either be bonded wholly with adhesives, wholly without adhesives, and/or some layers may be bonded with adhesives while other layers are bonded without adhesives.

Although FIG. 1A shows a depiction of a microfluidic device comprising one intermediate layer, it should be understood that greater than one (e.g., two, three, four, five, etc.) intermediate layers are possible. For example, in some embodiments, the one or more intermediate layers comprise a plurality of intermediate layers. FIG. 1B shows, according to some embodiments, a cross-sectional schematic diagram of microfluidic device 100b comprising first intermediate layer 104a and second intermediate layer 104b. In the depicted embodiment, plurality of channels 302, or other microfluidic components, may be formed by aligned portions of the patterns of removed material formed in the two intermediate layers. Specifically, the depicted channels are formed by the patterns of removed material being aligned such that the first intermediate layer defines a lower portion of each channel and the second intermediate layer defines an upper portion of each channel. The tops and bottoms of each channel 302 are defined by top layer 106 and substrate 102, respectively, and the intermediate layers are disposed between. However, instances in which the tops and/or bottoms of the channels, or other components defined by the intermediate layers, are formed by adjacent intermediate layers are also contemplated.

FIG. 1C shows, according to some embodiments, microfluidic device 100c, which is a modification of the embodiment of FIG. 1B where third intermediate layer 104c is disposed on first and second intermediate layers 104a and 104b between substrate 102 and top layer 106. Similar to the above, plurality of channels 302 are formed by aligned portions of the patterns of removed material formed in the first and second intermediate layers where the aligned portions are of the same size and shape. Additionally, a pattern of removed material in third intermediate layer 104c may form interconnecting channels 304 (e.g., interconnecting channels 304a and 304b) extending between two adjacent channels, or other features formed in one or more adjacent intermediate layers. As shown in the figure, the pattern of removed material in third intermediate layer 104c is sized and shaped such that a first portion of interconnecting channel 304 (e.g., 304a) formed in third intermediate layer 104c is in fluid communication with one or more first adjacent channels 302 and a second portion of interconnecting channel 304 (e.g., 304b) formed in third intermediate layer 104c is in fluid communication with one or more second adjacent channels 302 that are separate from the one or more first adjacent channels. This interconnecting channel 304 is defined by the patterned portions of third intermediate layer 104c disposed between top layer 106 and second intermediate layer 104b. Depending on the hydrophobicity of the first intermediate layer and/or the second intermediate layer, the flow of a liquid between the adjacent channels may either be facilitated (e.g., a hydrophilic surface exposed to the interior of the channel) and/or prevented (e.g., a hydrophobic surface exposed to the interior of the channel).

In view of the above, it should be understood that by stacking multiple intermediate layers on one another with desired patterns of removed material formed in each layer, it is possible to define multiple fluidly connected microfluidic components including microfluidic channels, valves, reservoirs, and/or any other appropriate microfluidic component that are located either in one or multiple intermediate layers of the device. Additionally, the formed components may either by hydraulic and/or pneumatic depending on the desired application and layout.

Depending on the size, shape, and/or surface properties of the patterns formed in the various layers, the substrate, intermediate layers, and/or top layer may be used to provide a number of different flow characteristics for the different components formed in a microfluidic device as disclosed herein. For example, in some embodiments, a substrate, intermediate layer, and/or top layer may be configured to facilitate fluid transport through one or more microfluidic channels of the microfluidic device. Referring to FIG. 1A, for example, substrate 102, which forms a bottom surface of plurality of microfluidic channels 302, may be configured to facilitate fluid transport through the one or more microfluidic channels 302 (e.g., by capillary action). In some embodiments, the one or more layers may be configured to facilitate fluid transport by being formed from or coated with a material that provides a hydrophilic surface exposed to the interior of the channel, or other microfluidic component, that may induce a capillary pressure that draws a liquid through the channel or other component. Of course, as elaborated on further below, different combinations of surface properties and relative proportions of the different patterns formed in the various layers may also be used to provide other functionalities.

Depending on the desired functionality, and the number of layers involved, the substrate, one or more intermediate layers, and top layer may either have the same or different hydrophobicities. For example, a substrate may have a first hydrophobicity, the one or more intermediate layers may have one or more second hydrophobicities, which may either be the same and/or different from one another, and the top layer may have a third hydrophobicity. In instances where it is desirable to retard the flow of a liquid through a particular feature, a layer with a larger hydrophobicity, i.e., more hydrophobic and less hydrophilic, may include a surface that is exposed to the interior volume of that portion of a microfluidic device. Alternatively, when it is desirable to promote the flow of a liquid through a particular feature, a layer with a smaller hydrophobicity, i.e., less hydrophobic and more hydrophilic, may include a surface that is exposed to the interior volume of the portion of the microfluidic device. Various combinations of different layers with different relative hydrophobicities are elaborated on in more detail below with regards to the disclosed embodiments.

As used herein, the term hydrophobic is given its ordinary meaning in the art and generally refers to a material that has a water contact angle greater than 90 degrees. Correspondingly, a hydrophilic material may generally refer to a material that has a water contact angle that is less than 90 degrees. In some embodiments, one or more capillary microfluidic elements require a certain range of contact angles to make them functional, as is described in further detail below.

It should be understood that the various substrates, intermediate layers, and top layer may be made from any appropriate material providing the desired functionalities for each layer. That said, in some embodiments, the substrate, one or more intermediate layers, and/or top layer of a microfluidic device may comprise: polymer films such as silicone, polyester, polypropylene, and other appropriate polymer films; copolymer films such as styrene-ethylene-butylene-styrene copolymer films, and/or polymers or other materials treated with hydrophilic or hydrophobic coatings. Additionally, the top layer and/or substrate of a microfluidic device as described herein may also be made from thicker non-film-based structures such as glass, bulk polymers, bulk copolymers, polymers or other materials treated with hydrophilic or hydrophobic coatings, photopolymers and/or resins used in three-dimensional (3D) printing, and/or any other appropriate material capable of functioning as a substrate and/or top layer for a microfluidic device as described herein. In some embodiments, it may be desirable to observe a flow of liquid through a microfluidic device. Accordingly, in some instances, the substrate, one or more intermediate layers, and/or top layer of a microfluidic device may be made from a transparent material.

As noted above, in some embodiments, a substrate, one or more intermediate layers, and/or a top layer may be made from a hydrophilic material. For example, hydrophilic polymers or other materials treated with hydrophilic coatings may be used. Additionally, the substrate and/or top layer may be made from a hydrophilic glass. Alternatively, the one or more layers of a microfluidic device may be made from a material that is modified to be hydrophilic. For instance, in some embodiments, a polymer film, or other structure depending on the particular component, may be coated with a hydrophilic material. Non-limiting examples of such materials include, but are not limited to, polymers coated with polyvinyl alcohol (PVA), a clear polyester film coated on one side with a hydrophilic coating (e.g., 3M™ 9984 Diagnostic Microfluidic Surfactant Free Fluid Transport Film); a double-sided clear polyester film coated on both sides with a hydrophilic coating (e.g., 3M™ Microfluidic Diagnostic Film 9960, 3M™ Microfluidic Diagnostic Film 9962); and/or a single-sided hydrophilic pressure-sensitive adhesive protected by a clear siliconized polyester release liner (e.g., ARflow® 93049 (Developmental)).

In addition to the above, a substrate, one or more intermediate layers, and/or a top layer may also comprise a hydrophobic material. Similar to the above, these layers may be made from a material that is inherently hydrophobic and/or a less hydrophobic material may be coated with a more hydrophobic material as the disclosure is not so limited. In certain embodiments, for example, the substrate, one or more intermediate layers, and/or top layer may be made from: a hydrophobic polymer such as polypropylene; polydimethylsiloxane (PDMS); a cyclic olefin copolymer (COC); poly(methyl methacrylate) (PMMA); photopolymers used in 3D printing; a hydrophobic copolymer such as styrene-ethylene-butylene-styrene; and/or any other appropriate hydrophobic material. Non-limiting examples of such materials include, but are not limited to, a single-sided silicone transfer adhesive with two polyester film release liners (e.g., ARclad® IS-8026); a double-sided polypropylene adhesive coated on both sides with a silicone pressure sensitive adhesive between two PET release liners (e.g., ARseal™ 90880); a styrene-ethylene-butylene-styrene film; a single-sided hydrophobic microfluidic diagnostic tape with delayed-tack adhesive (e.g., 9795R from 3M™); and/or a nitrile phenolic based thermosetting adhesive film (e.g., Thermal Bonding Film 583 from 3M™).

According to certain embodiments, the microfluidic device may comprise a plurality of intermediate layers, wherein at least one intermediate layer comprises a film and/or an adhesive (e.g., a polyester film, a pressure sensitive adhesive, etc.) and at least one intermediate layer comprises a polymer (e.g., polypropylene, PDMS, a COC, PMMA, etc.). Configuring the device in this way advantageously provides the ability to increase the thickness of the one or more intermediate layers that comprise a polymer, which may result in an increase in the overall liquid volume that the device is capable of handling (e.g., in the reservoirs and/or microfluidic channels of the one or more intermediate layers). In some embodiments, for example, the one or more intermediate layers comprising a polymer may be comparatively thick and used to define the microfluidic channels and reservoirs, while the one or more intermediate layers comprising a film and/or adhesive may be comparatively thin and used to define the functional capillary elements (e.g., valves). Suitable thicknesses of the intermediate layers are explained in further detail herein.

At least a portion of the substrate may be configured such that a side of the substrate facing the one or more intermediate layers has any of a variety of suitable (e.g., hydrophilic or hydrophobic) water contact angles. Referring to FIG. 1A, for example, surface 108b of substrate 102 facing intermediate layer 104 has any of a variety of suitable (e.g., hydrophilic or hydrophobic) contact angles. In some embodiments, the side of the substrate facing the one or more intermediate layers has a water contact angle greater than or equal to 5 degrees, greater than or equal to 10 degrees, greater than or equal to 15 degrees, greater than or equal to 20 degrees, greater than or equal to 25 degrees, greater than or equal to 30 degrees, greater than or equal to 35 degrees, greater than or equal to 40 degrees, greater than or equal to 45 degrees, greater than or equal to 50 degrees, greater than or equal to 55 degrees, greater than or equal to 60 degrees, greater than or equal to 65 degrees, greater than or equal to 70 degrees, greater than or equal to 75 degrees, greater than or equal to 80 degrees, greater than or equal to 85 degrees, greater than or equal to 90 degrees, greater than or equal to 95 degrees, greater than or equal to 100 degrees, greater than or equal to 105 degrees, greater than or equal to 110 degrees, greater than or equal to 115 degrees, greater than or equal to 120 degrees, greater than or equal to 125 degrees, greater than or equal to 130 degrees, greater than or equal to 135 degrees, greater than or equal to 140 degrees, greater than or equal to 145 degrees, greater than or equal to 150 degrees, greater than or equal to 155 degrees, greater than or equal to 160 degrees, greater than or equal to 165 degrees, or greater than or equal to 170 degrees. In certain embodiments, the side of the substrate facing the one or more intermediate layers has a water contact angle less than or equal to 180 degrees, less than or equal to 175 degrees, less than or equal to 170 degrees, less than or equal to 165 degrees, less than or equal to 160 degrees, less than or equal to 155 degrees, less than or equal to 150 degrees, less than or equal to 145 degrees, less than or equal to 140 degrees, less than or equal to 135 degrees, less than or equal to 130 degrees, less than or equal to 125 degrees, less than or equal to 120 degrees, less than or equal to 115 degrees, less than or equal to 110 degrees, less than or equal to 105 degrees, less than or equal to 100 degrees, less than or equal to 95 degrees, less than or equal to 90 degrees, less than or equal to 85 degrees, less than or equal to 80 degrees, less than or equal to 75 degrees, less than or equal to 70 degrees, less than or equal to 65 degrees, less than or equal to 60 degrees, less than or equal to 55 degrees, less than or equal to 50 degrees, less than or equal to 45 degrees, less than or equal to 40 degrees, less than or equal to 35 degrees, less than or equal to 30 degrees, less than or equal to 25 degrees, less than or equal to 20 degrees, less than or equal to 15 degrees, or less than or equal to 10 degrees. Combinations of the above recited ranges are also possible (e.g., the side of the substrate facing the one or more intermediate layers has a water contact angle greater than or equal to 5 degrees and less than or equal to 180 degrees, the side of the substrate facing the one or more intermediate layers has a water contact angle greater than or equal to 10 degrees and less than or equal to 40 degrees). Other ranges are also possible.

In certain embodiments, at least a portion of one or more of the intermediate layers and/or the top layer may have any of a variety of suitable (e.g., hydrophilic) water contact angles. In some embodiments, one or more of the intermediate layers and/or the top layer has a hydrophilic water contact angle greater than or equal to 5 degrees, greater than or equal to 10 degrees, greater than or equal to 15 degrees, greater than or equal to 20 degrees, greater than or equal to 25 degrees, greater than or equal to 30 degrees, greater than or equal to 35 degrees, greater than or equal to 40 degrees, greater than or equal to 45 degrees, greater than or equal to 50 degrees, greater than or equal to 55 degrees, greater than or equal to 60 degrees, greater than or equal to 65 degrees, greater than or equal to 70 degrees, greater than or equal to 75 degrees, greater than or equal to 80 degrees, or greater than or equal to 85 degrees. In certain embodiments, one or more of the intermediate layers and/or the top layer has a hydrophilic water contact angle less than or equal to 90 degrees, less than or equal to 85 degrees, less than or equal to 80 degrees, less than or equal to 75 degrees, less than or equal to 70 degrees, less than or equal to 65 degrees, less than or equal to 60 degrees, less than or equal to 55 degrees, less than or equal to 50 degrees, less than or equal to 45 degrees, less than or equal to 40 degrees, less than or equal to 35 degrees, less than or equal to 30 degrees, less than or equal to 25 degrees, less than or equal to 20 degrees, less than or equal to 15 degrees, or less than or equal to 10 degrees. Combinations of the above recited ranges are also possible (e.g., one or more of the intermediate layers and/or the top layer has a hydrophilic water contact angle greater than or equal to 5 degrees and less than or equal to 90 degrees, one or more of the intermediate layers and/or the top layer has a hydrophilic water contact angle greater than or equal to 10 degrees and less than or equal to 30 degrees). Other ranges are also possible.

In certain embodiments, at least a portion of the one or more of the intermediate layers and/or the top layer may have any of a variety of suitable (e.g., hydrophobic) water contact angles. In some embodiments, one or more of the intermediate layers and/or the top layer has a hydrophobic water contact angle greater than or equal to greater than or equal to 90 degrees, greater than or equal to 95 degrees, greater than or equal to 100 degrees, greater than or equal to 105 degrees, greater than or equal to 110 degrees, greater than or equal to 115 degrees, greater than or equal to 120 degrees, greater than or equal to 125 degrees, greater than or equal to 130 degrees, greater than or equal to 135 degrees, greater than or equal to 140 degrees, greater than or equal to 145 degrees, greater than or equal to 150 degrees, greater than or equal to 155 degrees, greater than or equal to 160 degrees, or greater than or equal to 165 degrees. In certain embodiments, one or more of the intermediate layers and/or the top layer has a hydrophobic water contact angle less than or equal to 170 degrees, less than or equal to 165 degrees, less than or equal to 160 degrees, less than or equal to 155 degrees, less than or equal to 150 degrees, less than or equal to 145 degrees, less than or equal to 140 degrees, less than or equal to 135 degrees, less than or equal to 130 degrees, less than or equal to 125 degrees, less than or equal to 120 degrees, less than or equal to 115 degrees, less than or equal to 110 degrees, less than or equal to 105 degrees, less than or equal to 100 degrees, or less than or equal to 95 degrees. Combinations of the above recited ranges are also possible (e.g., one or more of the intermediate layers and/or the top layer has a hydrophobic water contact angle greater than or equal to 90 degrees and less than or equal to 170 degrees, the side of the substrate facing the one or more intermediate layers has a hydrophobic water contact angle greater than or equal to 100 degrees and less than or equal to 110 degrees). Other ranges are also possible.

In the depicted embodiment, and in the other various embodiments described herein, each of the substrate, one or more intermediate layers, and top layer may have any of a variety of suitable thicknesses. In certain embodiments, for example, one or more layers may be relatively thin to sufficiently control fluid pressures, and one or more layers may be relatively thick to hold more liquid volume in the device. Referring to FIG. 1A, for example, the substrate 102, one or more intermediate layers 104, and top layer may have a thickness 110a, 110b, and 110c respectively. In some embodiments, the thicknesses of these layers may be greater than or equal to 1 micrometer, greater than or equal to 10 micrometers, greater than or equal to 20 micrometers, greater than or equal to 50 micrometers, greater than or equal to 100 micrometers, greater than or equal to 200 micrometers, greater than or equal to 300 micrometers, greater than or equal to 400 micrometers, greater than or equal to 500 micrometers, greater than or equal to 600 micrometers, greater than or equal to 700 micrometers, greater than or equal to 800 micrometers, greater than or equal to 900 micrometers, greater than or equal to 1 millimeter, or greater than or equal to 1.5 millimeters. In certain embodiments, the substrate, one or more intermediate layers, and top layer may also have a thickness less than or equal to 2 millimeters, less than or equal to 1.5 millimeters, less than or equal to 1 millimeter, less than or equal to 900 micrometers, less than or equal to 800 micrometers, less than or equal to 700 micrometers, less than or equal to 600 micrometers, less than or equal to 500 micrometers, less than or equal to 400 micrometers, less than or equal to 300 micrometers, less than or equal to 200 micrometers, less than or equal to 100 micrometers, or less than or equal to 50 micrometers. Combinations of the above recited ranges are also possible. For instance, a thickness of the individual layers of a device may be greater than or equal to 1 micrometer and less than or equal to 2 millimeters, greater than or equal to 50 micrometers and less than or equal to 200 micrometers, and/or any other appropriate combination of the foregoing ranges. According to some embodiments, the thickness of an individual layer of the device may be greater than 2 millimeters if larger volumes of liquid are needed to operate the microfluidic device. Other thicknesses of the various layers of the microfluidic devices including thicknesses both greater than and less than those noted above are also contemplated as the disclosure is not limited to any particular thickness of the individual layers.

As described above, in some embodiments, the thickness of one or more layers (e.g., one or more intermediate layers) may be comparatively thick relative to other layers of the microfluidic device. Configuring the device in this way advantageously allows for the ability to increase the overall volume of liquid that the microfluidic device is capable of handling. In some embodiments, for example, an intermediate layer comprising a polymer may define one or more microfluidic channels and/or one or more reservoirs of the device. In some embodiments, such an intermediate layer may be comparatively thick relative to other layers of the device in order to increase the volume of reservoirs and liquids running on the microfluidic device at any given time. The relatively thick intermediate layer may have any of a variety of suitable thicknesses. Without wishing to be bound by theory, the thickness of the one or more relatively thick intermediate layers may depend on the liquid volume required for operation of the microfluidic device, as the thickness of the one or more relatively thick intermediate layers does not have a significant effect on the capillary pressure of the microfluidic device, which is instead defined by one or more relatively thin intermediate layers. In some embodiments, for example, the relatively thick intermediate layer may have a thickness between greater than or equal to 500 micrometers and less than or equal to 2 millimeters. The relatively thinner layers of the device may, in some embodiments, define one or more functional capillary elements (e.g., valves). In certain embodiments, the relatively thin intermediate layer may have a thickness between greater than or equal to 1 micrometer and less than or equal to 2 millimeters, or a thickness between greater than or equal to 1 micrometer and less than or equal to 500 micrometers.

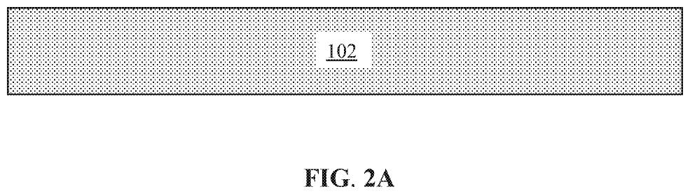

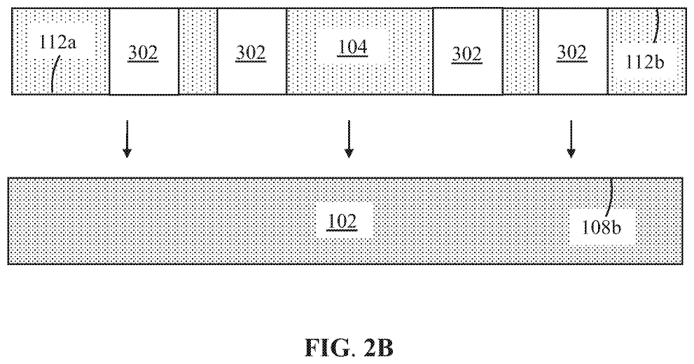

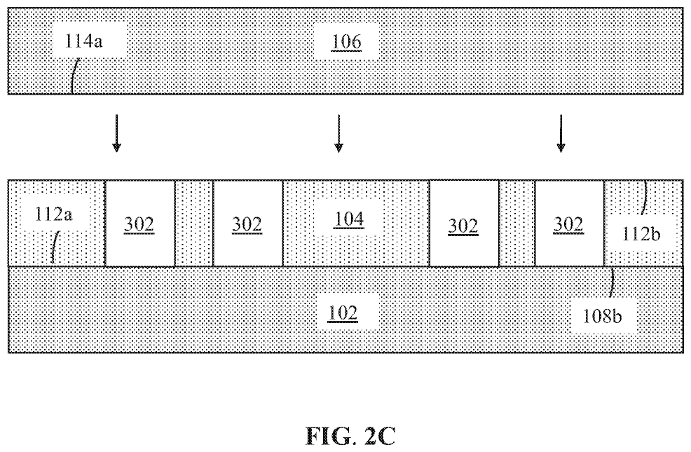

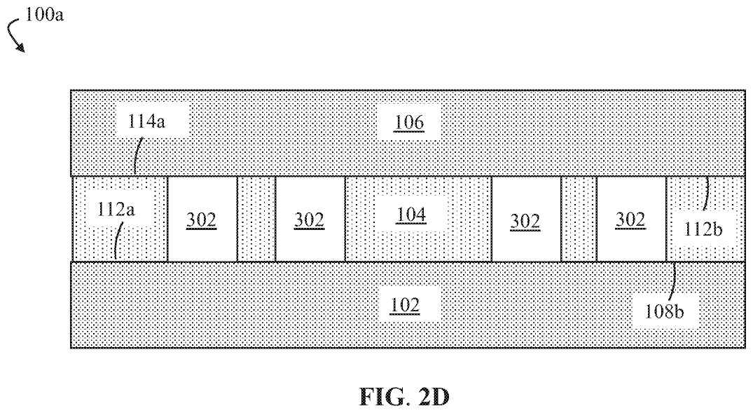

Having described the structures associated with FIGS. 1A-1C and related embodiments, a corresponding method of manufacturing a microfluidic device is described relative to FIGS. 2A-2D. The figures show a series of cross-sectional schematic diagrams depicting one embodiment of a manufacturing method of fabricating a microfluidic device.

In some embodiments, one or more intermediate layers are manufactured with a set of corresponding patterns where portions of material have been removed. At least portions of the patterns of the individual layers align with one another when stacked together such that the layers define a plurality of interconnected channels, reservoirs, valves, and/or other microfluidic components. The patterns of removed material in the intermediate layers may be formed, in some embodiments, by cutting, stamping, punching, and/or etching the patterns into the one or more intermediate layers. In certain non-limiting embodiments, for example, the patterns corresponding to the plurality of channels, reservoirs, valves, and/or other microfluidic components may be cut into the one or more intermediate layers using plot cutters and/or laser cutters, such as a Silhouette portrait craft cutter (Silhouette America, Lindon, UT) and/or a Graphtec Cutting Plotter CE-5000 (Graphtec America, Inc., Irvine, CA). Of course, any other appropriate method of forming a pattern in the one or more intermediate layers may also be used. In certain embodiments, for example, the patterns corresponding to the plurality of channels, reservoirs, valves, and/or other microfluidic components are formed in the one or more intermediate layers by 3D-printing. Different fabrication methods (e.g., cutting, stamping, punching, etching, 3D-printing, etc.) of the one or more intermediate layers can affect the surface properties of the layer. Accordingly, the capillary pressure can advantageously be tuned for a particular application by choosing the fabrication method.

After forming the desired patterns in the one or more intermediate layers, the method may include applying the one or more intermediate layers to a substrate. Referring, for example, to FIGS. 2A and 2B, intermediate layer 104 is positioned over substrate 102 in a desired position and orientation prior to placing the intermediate layer in contact with the substrate such that the intermediate layer is disposed on the substrate. While not depicted, in some embodiments, one or more additional intermediate layers may also be positioned over and placed into contact with the first intermediate layer in a desired position and orientation such that they are disposed on the first intermediate layer on a surface of the first intermediate layer opposite from the substrate. Thus, complex microfluidic structures defined by interconnected patterns formed in multiple intermediate layers may be constructed. As shown in FIG. 2C, in certain embodiments, the method also includes positioning a top layer over a topmost one of the one or more intermediate layers in a desired position and orientation. The top layer may then be placed into contact with the topmost one of the one or more intermediate layers such that the one or more intermediate layers are disposed between the topmost layer and the substrate as shown in FIG. 2D. Once assembled, the various microfluidic components such as channels, valves, reservoirs, and/other microfluidic components may be formed between the surfaces of the stacked substrate, one or more intermediate layers, and top layer.

As noted previously, the various layers of a microfluidic device may be held in a desired configuration using any number of different methods. For example, one or more adhesives may be placed between the various interfaces between the different layers. In one such embodiment, an adhesive may be disposed between the interfaces between the substrate and one or more intermediate layers, between adjacent intermediate layers, and/or between the top layer and a topmost one of the one or more intermediate layers. Depending on the type of adhesive used, different manufacturing methods may be implemented. For example, in the instance of a pressure sensitive adhesive, the act of pressing two layers together may bond the layers. Alternatively, in instances where a photo-curable adhesive is used, a light source that is at least partially transparent to the intervening layers of material may be applied to the stack of layers either after each layer is assembled and/or after all of the layers are assembled to cure the adhesive and bond the layers together. Of course, other appropriate types of adhesives may also be used.

In other embodiments, the various layers of a microfluidic device may be bonded to each other during an assembly process using other bonding methods. For example, in some embodiments, lamination (e.g., dry bond lamination), pressure, heat, and/or other methods capable of bonding the layers together may be used. Again, depending on the particular type of bonding method used, the various layers shown in FIGS. 2A-2C may be bonded together at each step in the assembly process and/or after the various layers have been stacked together as the disclosure is not limited in this fashion.

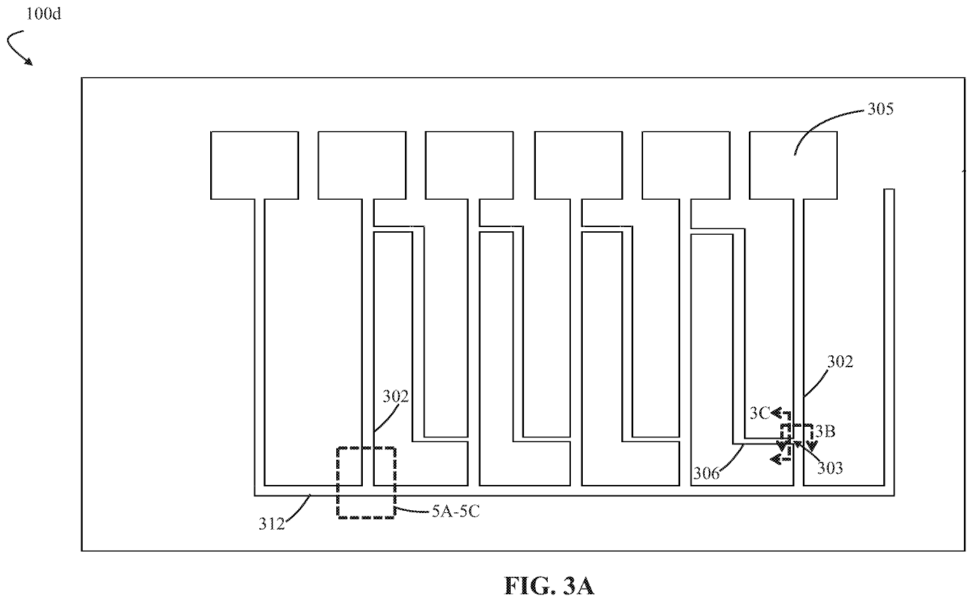

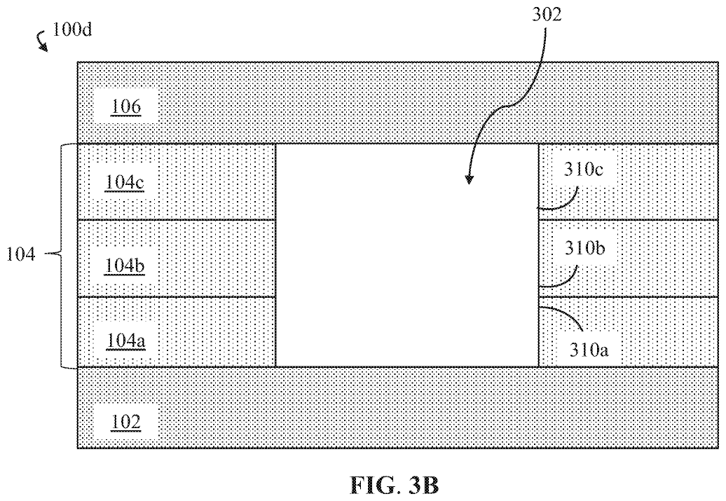

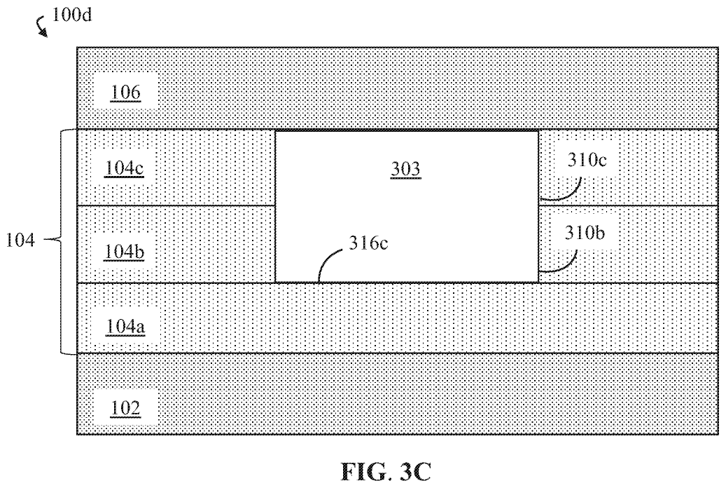

As explained above, the microfluidic device may comprise a plurality of microfluidic channels that are defined by patterns formed in the one or more intermediate layers. Referring, for example, to FIG. 3A, microfluidic device 100d comprises a plurality of microfluidic channels 302. The plurality of microfluidic channels may, in some embodiments, be hydraulic channels and/or pneumatic channels depending on the particular application and design of the device. The plurality of microfluidic channels may be configured such that each microfluidic channel has any of a variety of suitable shapes and sizes, as would be understood by a person of ordinary skill in the art. For example, in certain embodiments, one or more microfluidic channels have a length less than or equal to 20 mm. The length of the one or more microfluidic channels of the device may be straight and/or a serpentine pattern, depending on the desired length. The one or more microfluidic channels may have a width less than or equal to 2 mm and a height less than or equal to 1 mm (e.g., the thickness of the one or more intermediate layers). In some embodiments, the dimensions (e.g., length, width, etc.) of the one or more microfluidic channels may be larger as needed.

In certain embodiments, at least a portion of at least one microfluidic channel has a transverse cross-section that is orthogonal to a direction of flow through the at least one microfluidic channel. FIG. 3B shows, according to some embodiments, a cross-sectional schematic diagram along line 3B in FIG. 3A of first intermediate layer 104a, second intermediate layer 104b, and third intermediate layer 104c defining a microfluidic channel. As shown in FIG. 3A, the cross-sectional schematic diagram shown in FIG. 3B is taken upstream from an interface of microfluidic channel 302 with pneumatic channel 306 and valve 303.

As noted previously, the various layers of the microfluidic device may have different hydrophobicities relative to one another. Specifically, substrate 102, first intermediate layer 104a, second intermediate layer 104b, third intermediate layer 104c, and/or top layer 106 may either have the same and/or different hydrophobicities. Thus, unlike typical microfluidic systems which have a constant hydrophobicity along their transverse cross-section of the various components, the channels, valves, reservoirs, and/or other microfluidic components disclosed herein may have transverse cross-sections where at least a portion of the transverse cross section taken orthogonal to a direction of flow and/or an axis extending along a length of the microfluidic component may have a first hydrophobicity and a second portion of the transverse cross-section may have a second hydrophobicity that is different than the first hydrophobicity. Referring, for example, to FIG. 3B, first portion 310a of microfluidic channel 302, which is defined by first intermediate layer 104a, has a first hydrophobicity, second portion 310b, which is defined by second intermediate layer 104b, has a second hydrophobicity that is different than the first hydrophobicity, and third portion 310c, which is defined by third intermediate layer 104c, has a third hydrophobicity that is different from both the first and second hydrophobicities. Additionally, each of these hydrophobicities of the intermediate layers may be different from a hydrophobicity of the surfaces of substrate 102 and top layer 106 exposed to the interior volume of the channel.

Providing the different layers with different hydrophobicities may permit the construction of unique microfluidic components including, for example, stop valves to inhibit fluid flow and/or a series of trigger valves to facilitate fluid flow, depending on the configuration of the patterned features in the various layers and the corresponding hydrophobicities of the layers and/or features. One such embodiment is shown in FIG. 3C, which is a cross-sectional schematic diagram along line 3C in FIG. 3A of valve 303 located downstream from and in fluid communication with channel 302 at the interface of microfluidic channel 302 and pneumatic channel 306. In the depicted embodiment, first intermediate layer 104a has a greater hydrophobicity than substrate 102, and in some instances may be hydrophobic. In certain embodiments, second intermediate layer 104b may have an even greater hydrophobicity than first intermediate layer 104a, and third intermediate layer 104c may have yet an even greater hydrophobicity than second intermediate layer 104b. Thus, the channel formed between the top layer and the first intermediate layer by the pattern formed in the second and third intermediate layers may function as a stop valve due to the step change in height (e.g., relative to channel 302 shown and described in reference to FIG. 3B) and the increased hydrophobicity relative to the more hydrophilic substrate (and less hydrophobic first intermediate layer).

The stop valve described above with reference to FIG. 3C may also be positioned in other parts of the microfluidic device. Referring to FIG. 3A, for example, the stop valve described above may be positioned at the most downstream portion of a microfluidic channel, for example, at the interface of microfluidic channel 302 and primary channel 312. In some such embodiments, the stop valve may stop a fluid flowing downstream in microfluidic channel 302 from entering primary channel 312, at least until a certain capillary pressure is reached.

While a stop valve has been shown in the above embodiment, other microfluidic structures including trigger valves may also be formed depending on the particular sizes, shapes, and relative hydrophobicities of the various layers. In some embodiments, the first portion of the transverse cross-section of the microfluidic channel has a first hydrophobicity that is greater than the second hydrophobicity of the second portion of the transverse cross-section of the microfluidic channel. In some such embodiments, first portion 310a of microfluidic channel 302, at the interface of microfluidic channel 302 and valve 303, may function as a stop valve to inhibit fluid flow, and second portion 310b of microfluidic channel 302, at the interface of microfluidic channel 302 and valve 303, may function as a trigger valve to facilitate fluid flow. Additionally, while three intermediate layers have been depicted, any appropriate number of intermediate layers may be used as the disclosure is not limited in this fashion.

As noted above, in some embodiments, a transverse cross-section of a microfluidic component, such as a microfluidic channel, valve, reservoir, or other component, has at least first and second portions of the cross-section where the surfaces exposed to the interior of the microfluidic component have different hydrophobicities and/or hydrophilicities. Accordingly, it should be understood that the different hydrophobicities and/or hydrophilicities may have any appropriate relationship relative to one another depending on a desired application. Accordingly, a first hydrophobicity (or hydrophilicity) and a second hydrophobicity (or hydrophilicity) of different layers forming the transverse cross-section of a microfluidic component may exhibit any appropriate difference for a desired application. For example, in certain embodiments, a difference between the first and second hydrophobicities or hydrophilicities may be greater than or equal to 5%, 10%, 20%, 30%, 40%, 50%, 60%, 70%, 80%, 90%, and/or any other appropriate percentage of the larger hydrophobicity or hydrophilicity. Correspondingly, the difference between the first and second hydrophobicities or hydrophilicities may be less than or equal to 95%, 90%, 80%, 70%, 60%, 50%, 40%, 30%, 20%, 10%, or any other appropriate percentage of the larger hydrophobicity or hydrophilicity. Combinations of the above recited ranges are contemplated including, for example, a difference between the first and second hydrophobicities or hydrophilicities that is between or equal to 5% and 95% of the larger hydrophobicity or hydrophilicity. However, other combinations of the foregoing ranges as well as differences between the first and second hydrophobicities or hydrophilicities both greater than and less than those noted above are also contemplated as the disclosure is not so limited.

As would be understood by a person of ordinary skill in the art, the first and second portions of the transverse cross-section of the microfluidic component having different hydrophobicities or hydrophilicities may also have different water contact angles. In some embodiments, for example, the first portion of the microfluidic component may have a first water contact angle and the second portion of the microfluidic component may have a second water contact angle, wherein a difference between the first and the second water contact angle is greater than or equal to 5%, 10%, 20%, 30%, 40%, 50%, 60%, 70%, 80%, or 90%, or wherein a difference between the first and second water contact angle is less than or equal to 95%, 90%, 8%, 70%, 60%, 50%, 40%, 30%, 20%, or 10%. The water contact angle of the first portion and/or the second portion may, in some embodiments, be greater than or equal to 10 degrees and less than or equal to 180 degrees.

In certain embodiments, the first and second hydrophobicities or hydrophilicities of the first portion and the second portion of the microfluidic component may be the same or substantially the same, as the disclosure is not meant to be limiting in this regard.

According to some embodiments, a stop valve may be defined by more than one layer of the microfluidic system (e.g., a substrate and/or one or more intermediate layers, one or more intermediate layers) such that the surface and side walls of the microfluidic channel vary in hydrophobicities or hydrophilicities due to the surface being defined by a first layer (e.g., with a first hydrophobicity or hydrophilicity) and the side walls being defined by one or more second layers (e.g., with at least a second hydrophobicity or hydrophilicity that is different from the first hydrophobicity or hydrophilicity). In certain embodiments, for example, a bottom surface of a microfluidic channel defined by, for example, the first hydrophilic layer may be hydrophilic (or less hydrophobic), and the side walls of the microfluidic channel defined by, for example, the one or more second layers may be more hydrophobic (or less hydrophilic) than the surface of the first layer exposed to an interior of the channel. In some embodiments, while the hydrophilic (or less hydrophobic) bottom surface may be configured to facilitate fluid flow, the hydrophobic (or less hydrophilic) side walls advantageously stop liquid flow, therefore functioning as a stop valve.

In certain embodiments, for example, a bottom surface of at least a portion of at least one microfluidic channel may have a first hydrophilicity or hydrophobicity. In some embodiments, the bottom surface of at least portion of at least one microfluidic channel may be hydrophilic. The bottom surface of the portion of the at least one microfluidic channel may, in some embodiments, be defined by an intermediate layer. Referring to FIG. 3C, for example, bottom surface 316c of microfluidic channel 302 is defined by first intermediate layer 104a, which may, in some embodiments, be hydrophilic. According to some embodiments, one or more side walls of at least a portion of the at least one microfluidic channel may have a second hydrophilicity or hydrophobicity that is greater than the first hydrophilicity or hydrophobicity. The second hydrophilicity or hydrophobicity may be at least 5% greater, 10% greater, 15% greater, 20% greater, 35% greater, 40% greater, 45% greater, or more than the first hydrophilicity or hydrophobicity. In some embodiments, the one or more side walls of at least a portion of the at least one microfluidic channel may be hydrophobic. The one or more side walls of the portion of the at least one microfluidic channel may, in certain embodiments, be defined by one or more intermediate layers. Referring to FIG. 3C, for example, second portion 310b of the side wall of microfluidic channel 302 and third portion 310c of the side wall of microfluidic channel 302 may be defined by second intermediate layer 104b and third intermediate film layer 104c, respectively, which may, in certain embodiments, be hydrophobic (or less hydrophilic). As described herein, second portion 310b and portion 310c may have different hydrophobicities, in some embodiments. In other embodiments, second portion 310b and portion 310c may have the same hydrophobicity. In certain embodiments, although not shown in the figures, the bottom surface of the portion of the microfluidic channel may be defined by a first intermediate layer and the one or more side walls of the portion of the microfluidic channel may be defined by a second intermediate layer, as the disclosure it not meant to be limiting in this regard.

According to some embodiments, a bottom surface of at least a portion of at least one microfluidic channel may be defined by a substrate. Referring, for example, to FIG. 1A, bottom surface 316a of microfluidic channel 302 is defined by substrate 102, which may, in some embodiments, be hydrophilic. In certain embodiments, one or more side walls of at least a portion of the at least one microfluidic channel may be defined by an intermediate layer. Referring to FIG. 1A, for example, portion 310d of the side wall of microfluidic channel 302 may be defined by intermediate layer 104, which may, in some embodiments, be hydrophobic. In other embodiments, the one or more side walls of at least a portion of the at least one microfluidic channel may be defined by more than one intermediate layer. Referring, for example, to FIG. 1B, portion 310e of the side wall of microfluidic channel 302 may be defined by intermediate layer 104a and portion 310f of the side wall of microfluidic channel 302 may be defined by intermediate layer 104b. In certain embodiments, portion 310c and portion 310f may have the same or different hydrophobicities.

According to certain embodiments, a single intermediate layer of the device may be configured to function as a stop valve by modifying the surface properties of a portion of the intermediate layer. In some embodiments, for example, a portion of the intermediate layer may be patterned to change the degree of hydrophilicity or hydrophobicity of the portion relative to the remainder of the microfluidic channel such that the portion can function as a stop valve.

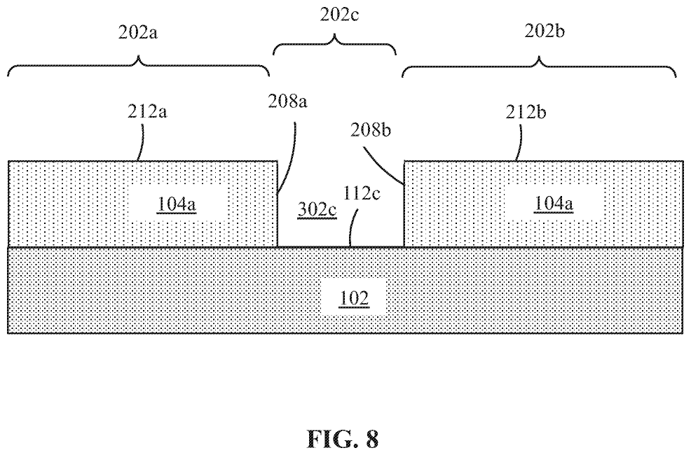

FIG. 8 shows, according to some embodiments, a cross-sectional schematic diagram of microfluidic channel 302c having three portions: first portion 202a and second portion 202b are defined by first intermediate layer 104a, while third portion 202c is disposed between first portion 202a and second portion 202b. One or more surfaces of first portion 202a, such as top surface 212a, may have a different hydrophobicity (e.g., more hydrophilic) than one or more surfaces of third portion 202c, such as side walls 208a and/or bottom surface 112c. One or more surfaces of second portion 202b, such as top surface 212b, may have a different hydrophobicity (e.g., more hydrophilic) than one or more surfaces of third portion 202c, such as side wall 208b and/or bottom surface 112c. Additionally, each of these hydrophobicities of the intermediate layer may be different from a hydrophobicity of the surfaces of substrate 102 and top layer 106 (not shown in FIG. 8) exposed to the interior volume of microfluidic channel 302c.

In certain embodiments, a bottom surface of at least a portion of at least one microfluidic channel may have a first hydrophobicity. In some embodiments, the bottom surface of at least a portion (e.g., the middle portion) of the at least one microfluidic channel may be hydrophobic. For example, the bottom surface of the portion of the at least one microfluidic channel may, in some embodiments, be defined by a substrate. According to some embodiments, one or more side walls of the intermediate layer may have a second hydrophobicity that is more hydrophobic than the bottom surface of the portion of the at least one microfluidic channel. For example, the second hydrophobicity may be at least 5% greater, 10% greater, 15% greater, 20% greater, 35% greater, 40% greater, 45% greater, or more than the first hydrophobicity. In some embodiments, the one or more side walls of the portion of the at least one microfluidic channel may, in certain embodiments, be defined by one or more intermediate layers.

In some embodiments, the sides and/or other surfaces of the layers and/or channels can be modified to be either more or less hydrophobic using any appropriate method including inherent material properties, surface treatments, laser etching, and/or any other appropriate type of treatment to provide a desired surface property for one or more portions of the microfluidic devices described herein.

As described herein, a microfluidic device may, in some embodiments, comprise a microfluidic channel having a gap or area of increased hydrophobicity in between two separated portions of the channel. For example, in certain embodiments, a channel has a middle portion disposed between two opposing portions of the channel extending along a length of the channel, wherein the middle portion isolates liquids separately contained in one or both of the opposing portions of the channel. Such a configuration may, in some embodiments, be fabricated using individual layers of films to form different portions of the microfluidic channel, as described in further detail below.

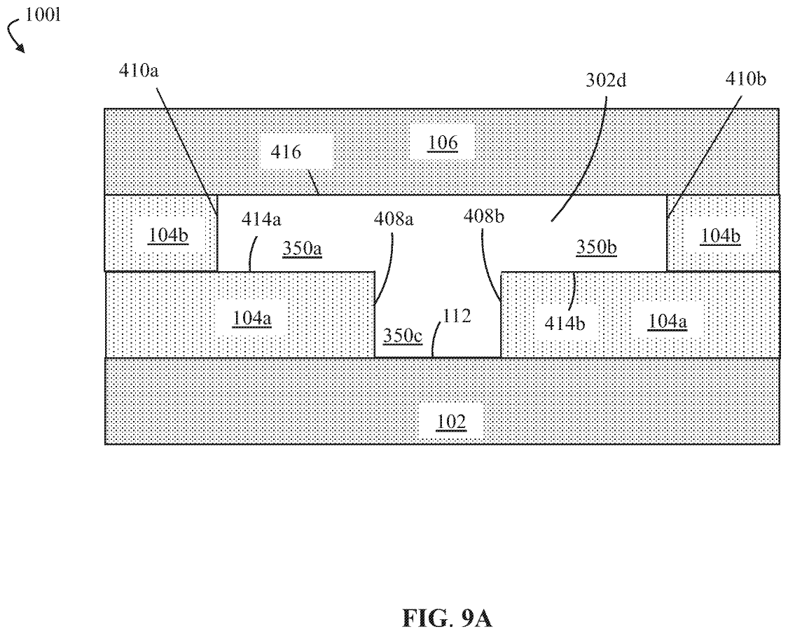

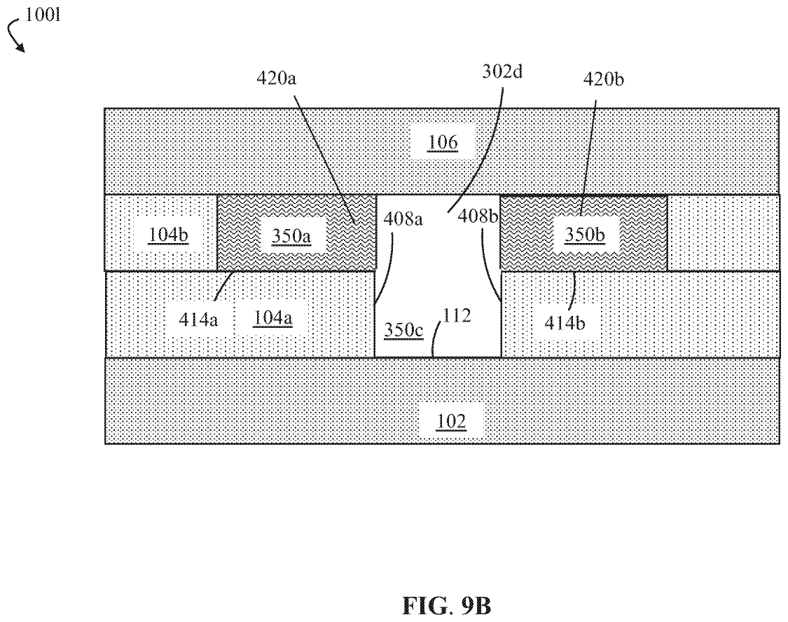

FIG. 9A shows a cross-sectional schematic diagram of microfluidic device 100l, according to some embodiments. Device 100l comprises substrate 102, first intermediate layer 104a disposed on substrate 102, and second intermediate layer 104b disposed on first intermediate layer 104a opposite from substrate 102. Top layer 106 is disposed on second intermediate layer 104b opposite substrate 102, defining channel 302d between top layer 106 and substrate 102. It should be understood that the substrate and top layer may comprise either the same or different materials relative to each other and the intermediate layers as the disclosure is not so limited. First intermediate layer 104a includes a cut out, or recessed portion, that forms a recessed channel corresponding to third portion 350c of channel 302d. Correspondingly, first intermediate layer 104a includes two opposing top surfaces 414a and 414b exposed to channel 302d and oriented towards top layer 106 on either side of the recessed channel corresponding to third portion 350c. Thus, the overall channel includes a primary channel with first portion 350a above top surface 414a and second portion 350b above surface 414b disposed on opposing sides of the recessed portion 350c of channel 302d.

As shown in FIG. 9A, the recessed portion of channel 302d, i.e., third portion 350c, may include bottom surface 112 and side walls 408a and 408b extending upwards from the bottom surface. The side walls may be defined by a respective thickness of first intermediate layer 104a. Bottom surface 112 may be a surface of substrate 102 oriented towards top layer 106, although it should be appreciated that additional layers or surface modification may be applied to the surface of substrate 102 to form bottom surface 112 of the recessed portion (i.e., third portion 350c) of channel 302d. Channel 302d thus consists of a recessed channel and a primary channel in the space enclosed between substrate 102, first intermediate layer 104a, second intermediate layer 104b, and top layer 106. Of course, while individual layers have been shown, it should be understood that the various layers may correspond to any appropriate number of intermediate layers including, for example, a plurality of intermediate layers stacked together to form each of the indicated layers as the disclosure is not so limited.

Surfaces in channel 302d may exhibit different hydrophobicities such that one or more fluids may be isolated in the first and second portions 350a and 350b of the channel without entering the recessed portion of the channel (i.e., third portion 350c) and/or without intermixing with each other, based on capillary pressure. This can be accomplished by having at least one surface of the third portion of the channel be more hydrophobic than adjacent surfaces of the first portion of the channel and the second portion of the channel. For example, at least side wall 408a may be more hydrophobic than surface 414a. Alternatively, or additionally, at least side wall 408b may be more hydrophobic than surface 414b. However, to permit the two volumes to be intermixed, it may also be desirable to facilitate the flowing of a liquid through the recessed portion of the channel. Thus, in some embodiments, bottom surface 112 of the recessed portion of the channel may be less hydrophobic (i.e., more hydrophilic) than the surfaces of side walls 408a and 408b of the recessed portion of the channel. This may permit a liquid to be selectively flowed through the recessed portion of the channel to permit intermixing of liquids contained in the isolated portions of the primary channel as elaborated on below. Accordingly, one or more liquids may be confined within the first and second portions 350a and 350b without entering the recessed portion, i.e., third portion 350c, of channel 302d. In effect, a stop valve is created to pin fluids in the first and second portions of channel 302d based on the capillary pressure due to the difference in hydrophobicity between surfaces in the recessed portion of the channel and adjacent surfaces in the primary channel prior to use.

It should be appreciated that while exemplary constructions of capillary stop valves fabricated using layers of films are discussed above in reference to FIG. 9A, aspects of the present disclosure are not so limited to such construction. For example, other construction techniques familiar to a person skilled in the field of microfluidic devices such as but not limited to additive manufacturing, subtractive manufacturing, and surface treatments may be used to fabricate a device similar to that shown in the figures without the use of intermediate film layers. Accordingly, the disclosed microfluidic devices and corresponding methods are not limited to only being used with constructions including intermediate film layers.

FIG. 9B shows the cross-sectional schematic diagram of the microfluidic device in FIG. 9A, with pinned fluids in first and second portions 350a and 350b, respectively, of channel 302d, in accordance with some embodiments. Pinned fluids 420a and 420b are disposed in respective first and second primary portions 350a and 350b of channel 302d, while surfaces in the recessed portion (i.e., third portion 350c) of channel 302d create sufficient capillary pressure to isolate fluids 420a and 420b from each other, such that no intermixing between the two fluids can occur. When it is desired to intermix the two fluids 420a and 420b, a third fluid (not shown) can be flowed through the recessed portion of the channel, which can provide affinity between the two fluids 420a and 420b to merge the three fluids within the channel 302d. Thus, intermixing of isolated first and second substances disposed in the isolated portions of the primary channel may be triggered by flowing a liquid through the recessed portion of the channel.

While liquids are illustrated in the two isolated portions of the primary channel, dried solids, such as one or more dried reagents, may also be disposed in one of the isolated portions of the primary channel and a liquid may be disposed in the other isolated portion of the primary channel such that upon intermixing of the volumes, the one or more dried reagents may be reconstituted in an even fashion along a length of the channel. For example, in some embodiments, one or more surfaces such as top surfaces 414a and 414b in the primary channel as shown in FIG. 9A may be configured to hold dried reagents which can be stable in ambient conditions for longer periods of time as compared to liquid reagents. The dried reagents can be reconstituted into a solution after introduction of liquids into the isolated portion of the channel holding the dried reagents.

Some aspects of the present disclosure are directed to a method of operating a microfluidic device to perform functions such as reconstitution of dried reagents, dilution and mixing of reagents, and others. In some embodiments, a microfluidic device similar to those shown in FIGS. 9A and 9B is used to hold two substances in two or more isolated portions of a primary channel. A liquid flowed through a recessed channel disposed between these isolated portions can be used to merge the two volumes and enable mixing of the substances.

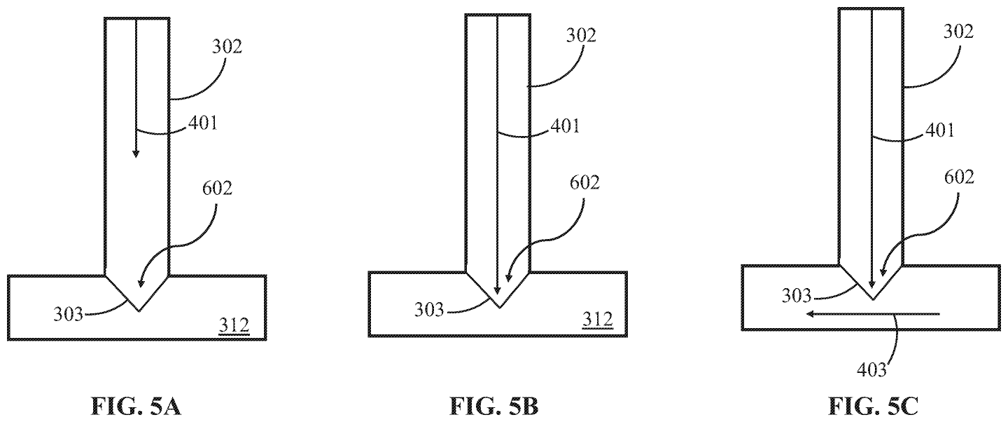

In some embodiments, an intermediate layer is configured to at least partially define a first microfluidic channel and a second microfluidic channel that intersect with each other. Sec, for example, FIGS. 5A-5C, which are top-view schematic diagrams of portion 5A-5C in FIG. 3A. Referring to FIG. 5A, a first microfluidic channel (e.g., channel 302) and a second microfluidic channel (e.g., primary channel 312) may intersect with each other, in some embodiments. In certain embodiments, the first microfluidic channel (e.g., channel 302) and the second microfluidic channel (e.g., primary channel 312) may be defined by a single intermediate layer.

According to some embodiments, an interface between the first microfluidic channel and the second microfluidic channel, which in the depicted embodiment corresponds to a portion of the second microfluidic channel adjacent to the intersection of the first channel and the second channel, may be patterned such that the interface is configured to function as a stop valve. Referring again to FIG. 5A, interface 602 between the first microfluidic channel (e.g., channel 302) and the second microfluidic channel (e.g., primary channel 312) may be patterned such that the interface is configured to function as stop valve 303. The interface between the first microfluidic channel and the second microfluidic channel may advantageously function as a stop valve due to a change in the degree of hydrophilicity or hydrophobicity at the interface resulting from the patterned design. In some embodiments, for example, interface 602 has a hydrophobicity that is greater than the hydrophobicity of the surrounding portions of the first microfluidic channel (e.g., channel 302) and/or the second microfluidic channel (e.g., primary channel 312). In some embodiments, for example, the interface between the first microfluidic channel and the second microfluidic channel may have a hydrophobicity that is at least 5% greater, 10% greater, 15% greater, 20% greater, 35% greater, 40% greater, 45% greater, or more than the surrounding portions of the first microfluidic channel and/or the second microfluidic channel.

According to some embodiments, the water contact angle at the interface may, in some embodiments, be higher than the water contact angle of the first microfluidic channel (e.g., channel 302) and/or the second microfluidic channel (e.g., primary channel 312) to be able to stop a liquid flow and function as a stop valve (e.g., stop valve 303). In certain embodiments, the value of the water contact angle can vary with different liquids and the hydrophobicity of the interface may be adjusted accordingly.

The interface between the first microfluidic channel and the second microfluidic channel may be patterned using any of a variety of suitable means to increase a hydrophobicity and/or water contact angle of the surface corresponding to the interface relative to adjacent portions of the second microfluidic channel. In certain embodiments, for example, the interface between the first microfluidic channel and the second microfluidic channel is laser etched and/or engraved. In some embodiments, cutting one or more thin cuts through the interface may form a hydrophobic barrier that can stop liquid advancement, therefore allowing the interface to function as a stop valve.

In some embodiments, the surface of the microfluidic channel (e.g., channel 302) may have a first hydrophobicity or hydrophilicity, and patterning the interface by laser etching and/or engraving causes the interface to have a second hydrophobicity or hydrophilicity that is different from the first hydrophobicity or hydrophilicity of the surface of the remainder of the channel. Referring, for example, to FIG. 5A, a liquid may flow in first flow direction 401 of microfluidic channel 302 due to the surface of microfluidic channel 302 being rendered hydrophilic. When the liquid reaches interface 602, as shown in FIG. 5B, the liquid may stop at stop valve 303 due to a change in hydrophilicity as a result of patterning interface 602 by laser etching and/or engraving. In certain embodiments, for example, interface 602 may be hydrophobic (or less hydrophilic).

The interface between the first microfluidic channel and the second microfluidic channel may have any of a variety of suitable patterned designs. In some embodiments, for example, and as shown in FIGS. 5A-5C, interface 602 comprises a triangular pattern. Other patterns are also possible, however, as the disclosure is not meant to be limiting in this regard. In some embodiments, for example, circular (e.g., semicircular) patterns may be used at the interface.

In certain embodiments, the stop valve formed at the interface of the first microfluidic channel and the second microfluidic channel may be used to merge the first fluid stopped at the stop valve with a second fluid. Referring, for example, to FIG. 5C, the first liquid flowing in first flow direction 401 of the first microfluidic channel (e.g., channel 302) and stopping at stop valve 303 may merge with a second liquid flowing in second flow direction 403 of the second microfluidic channel (e.g., primary channel 312). The stop valve formed at the interface may therefore advantageously allow the user to control the timing and/or degree of mixing between the first fluid (e.g., stopped at the stop valve) and the second fluid when the second fluid flows through the second channel triggering the illustrated stop valve.

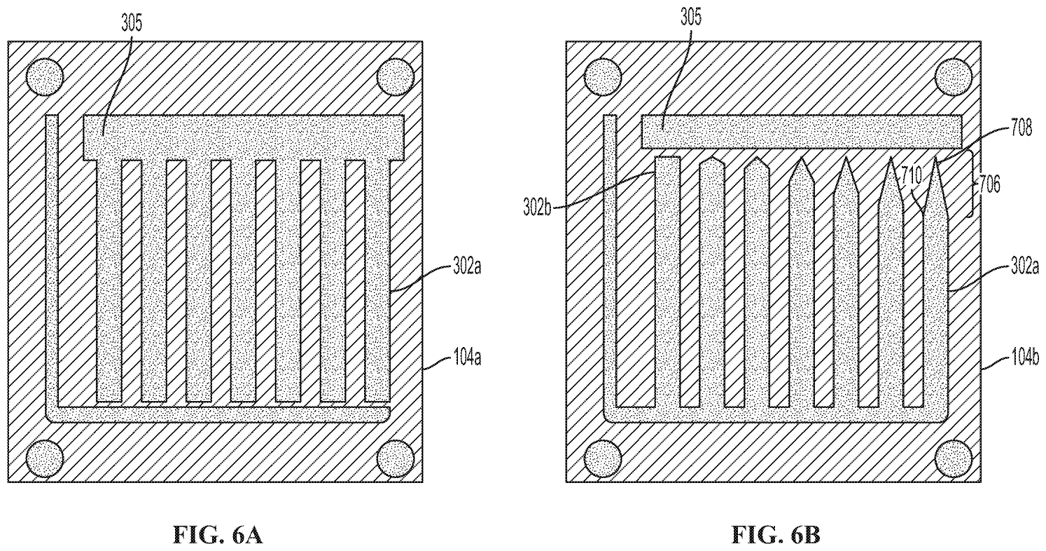

The capillary pressure of the microfluidic channels may be controlled in ways other than just tuning the hydrophobicity and/or hydrophilicity of portions of the microfluidic channels. In some embodiments, for example, changing the geometry and/or dimensions of a microfluidic channel may also cause the channel to act as a valve (e.g., a retention valve, a stop valve). As explained herein, a plurality of intermediate layers may be configured to at least partially define a plurality of fluidly connected microfluidic components (e.g., channels, reservoirs, and/or valves). In certain embodiments, a portion of at least one microfluidic channel is fluidly connected to and proximate a reservoir. See, for example, FIGS. 6A and 6B, which show first intermediate layer 104a and second intermediate layer 104b, respectively, that at least partially define at least one microfluidic channel 302 and at least one inlet reservoir 305. In some embodiments, for example, first intermediate layer 104a at least partially defines microfluidic channel 302a and a portion of second intermediate layer 104b is exposed to an interior of a portion of microfluidic channel 302a. As shown in FIGS. 6A and 6B, microfluidic channels 302 are fluidly connected to and proximate inlet reservoir 305.

According to some embodiments, second intermediate layer 104b may be disposed on top of first intermediate layer 104a. Second intermediate layer 104b may be a hydrophobic layer such that second intermediate layer 104b is more hydrophobic than first intermediate layer 104a, in certain embodiments. For example, second intermediate layer 104b may have a hydrophobicity that is at least 5% greater, 10% greater, 15% greater, 20% greater, 35% greater, 40% greater, 45% greater, or more than first intermediate layer 104a.

In certain embodiments, first intermediate layer 104 and second intermediate layer 104b may have the same hydrophobicity or hydrophilicity, however the water contact angle of one or more portions and/or components of first intermediate layer 104a and/or second intermediate layer 104b may be adjusted to stop and/or facilitate the flow of liquid through the one or more portions and/or components of first intermediate layer 104a and/or second intermediate layer 104b.

In some embodiments, a portion of second intermediate layer 104b (e.g., hydrophobic layer) exposed to an interior of a portion of microfluidic channel 302 has a first transverse dimension at an upstream location and a second transverse dimension at a downstream location, wherein the first transverse dimension is smaller than the second transverse dimension. Referring, for example, to FIG. 6B, portion 706 of second intermediate layer 104b exposed to an interior of microfluidic channel 302 has first transverse dimension 708 at an upstream location of microfluidic channel 302 that is smaller than second transverse dimension 710 at a downstream location of microfluidic channel 302. In some embodiments, the first transverse dimension exposed to the interior portion of microfluidic channel 302 may be at an inlet of microfluidic channel 302 that is fluidly connected to and proximate inlet reservoir 305.

Advantageously, such a gradual change from a smaller, first transverse dimension to a larger, second transverse dimension may, in some embodiments, result in the portion of the at least one microfluidic channel functioning as a retention valve. In certain embodiments wherein the second intermediate layer is a hydrophobic layer that is more hydrophobic than the first intermediate layer, for example, the transition from the first transverse dimension to the second transverse dimension results in a liquid in the microfluidic channel being exposed to a larger surface area of hydrophobic material at an upstream location (e.g., at the inlet) of the microfluidic channel that transitions to a smaller surface of hydrophobic material area at a downstream location of the microfluidic channel. The pressure gradient (e.g., capillary pressure gradient) at the first transverse dimension may be greater than the pressure gradient at the second transverse dimension, resulting in portion 706 of microfluidic channel 302a functioning as a retention valve for a liquid in microfluidic channel 302a. In some non-limiting embodiments, a triangular geometry, as shown in FIG. 6B, for example, helps in facilitating the liquid flow and crossing the hydrophobic barrier by coalescing two adjacent menisci at first transverse dimension 708.

Any of a variety of suitable dimensions may be employed for the first transverse dimension and the second transverse dimension as long as the portion of the hydrophobic layer exposed to the interior of the microfluidic channel is configured such that a first, comparatively smaller transverse dimension transitions into a second, comparatively larger transverse dimension. FIG. 6B shows, for example, a plurality of microfluidic channels with a gradient change in dimensions from microfluidic channel 302b to microfluidic channel 302a. Although the figures show a triangular dimension, any of a variety of suitable dimensions may be employed, as the disclosure is not meant to be limiting in this regard.

As explained herein, the fluidly connected microfluidic components may be defined by more than two intermediate layers (e.g., three intermediate layers). Although FIGS. 6A-6B show only two intermediate layers, there may be additional intermediate layers (e.g., between first intermediate layer 104a and second intermediate layer 104b) that define the fluidly connected microfluidic components.

As described herein, a microfluidic device may, in some embodiments, comprise a microfluidic channel having a transverse cross-section that is orthogonal to a direction of flow through the microfluidic channel, wherein a first portion of the transverse cross-section has a first channel height, and a second portion of the transverse cross-section has a second channel height that is different from the first channel height. Such a configuration may, in some embodiments, be fabricated using individual layers of films to form different portions of the microfluidic channel, as described in further detail below.

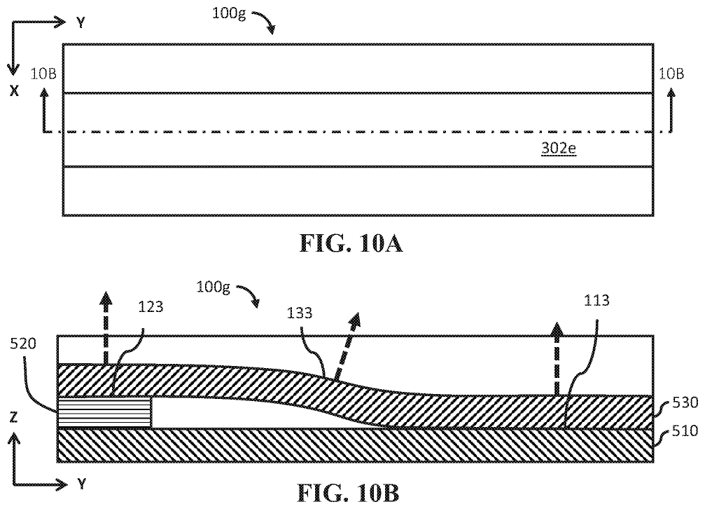

FIG. 10A depicts one embodiment of microfluidic device 100g, and FIG. 10B is a longitudinal cross-section of microfluidic device 100g of FIG. 10A. Microfluidic device 100g includes microfluidic channel 302e, only a portion of which is shown in FIGS. 10A and 10B. The microfluidic channel extends in a first direction aligned with the Y direction shown in FIGS. 10A and 10B. Microfluidic device 100g comprises a plurality of layers, including first layer 510, second layer 520, and third layer 530. Other embodiments of a microfluidic device may include more or fewer layers, as the present disclosure is not limited in this regard. In some embodiments, the first layer may be a substrate layer. In some embodiments, one or more of the layers may be a film. In the embodiment of FIGS. 10A and 10B, second layer 520 is disposed on first layer 510, and third layer 530 is disposed on second layer 520. Additionally, a portion of third layer 530 is directly disposed on first layer 510. In some embodiments, third layer 530 is adhered to first layer 510, such as with the use of an adhesive.

Third layer 530 is disposed on first layer 510 at first location 113, and third layer 530 is disposed on second layer 520 at second location 123 which may be located upstream from the first location along a length of the channel. Third layer 530 is inclined in region 133 between first location 113 and second location 123. A region of a layer of a microfluidic device may be inclined relative to another portion of the microfluidic device. For example, referring to FIG. 10B, third layer 530 includes a top surface in first location 113, wherein the top layer is generally planar and extends in the X and Y directions. As such, the top surface of third layer 530 in first location 113 includes a surface normal that extends in the Z direction. Similarly, third layer 530 includes a top surface in second location 123, wherein the top layer is generally planar and extends in the X and Y directions. As such, the top surface of third layer 530 in second location 123 includes a surface normal that extends in the Z direction. In region 133 between first location 113 and second location 123, third layer 530 is inclined such that a top surface of third layer 530 in region 133 includes a surface normal that is angled relative to the Z direction (surface normals are indicated in FIG. 10B with bold, dashed-line arrows). Stated differently, a surface of the inclined region 133 is angled relative to a surface of third layer 530 at first location 113. Similarly, a surface of the inclined region 133 is angled relative to a surface of third layer 530 at second location 123. The inclination angle of the inclined region may be any appropriate angle. The inclination angle may also vary as a function of position. For example, the inclination angle may increase from a small value near first location 113 to a maximum value, and then decrease again near second location 123. In some embodiments, an inclination angle (e.g., a maximum inclination angle) may be greater than 0 degrees and less than 89 degrees, between or equal to 1 degree and 89 degrees, between or equal to 5 degrees and 89 degrees, and/or any other appropriate range of angles.

In the embodiment of FIGS. 10A and 10B, inclined region 133 is inclined in the direction in which microfluidic channel 302e extends. That is, microfluidic channel 302e extends in a first direction aligned with the Y direction, and inclined region 133 is angled at least partially in the first direction (i.e., in a direction aligned with the Y direction). In other embodiments, an inclined region may be angled in other directions. For example, an inclined region may be angled at least partially in a second direction transverse to the first direction, as described in greater detail below in reference to other figures. In the embodiment of FIGS. 10A and 10B, a capillary pressure gradient is aligned with the direction in which microfluidic channel 302e extends, due to the height of microfluidic channel 302e varying along the length of microfluidic channel 302e. As such, the capillary pressure gradient may increase along a length of the inclined region, thus urging fluid to be retained at high capillary pressure regions and flow in the direction of the pressure gradient to low capillary pressure regions (e.g., the negative Y direction as depicted in FIGS. 10A and 10B).

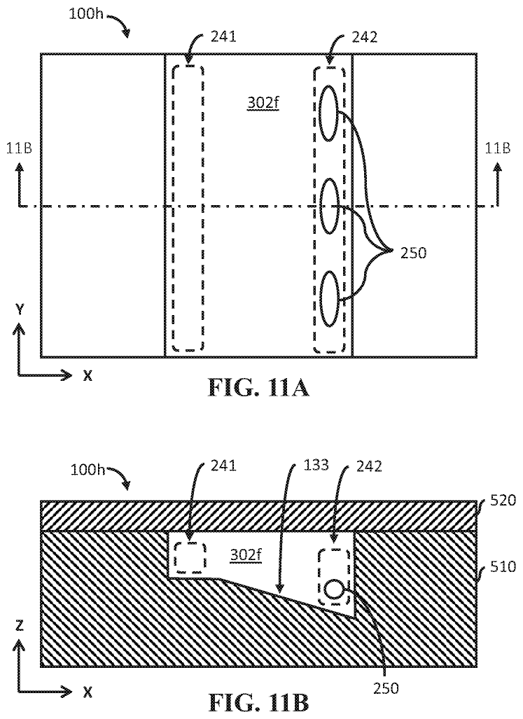

FIG. 11A depicts one embodiment of microfluidic device 100h, and FIG. 11B is a transverse cross-section of microfluidic device 100h of FIG. 11A. Microfluidic device 100h includes microfluidic channel 302f extending in a first direction aligned with the Y direction of FIGS. 11A and 11B. Microfluidic device 100h comprises a plurality of layers, including first layer 510 and second layer 520. As elaborated on further below, the first layer may include a plurality of layers in some embodiments. In the embodiment of the figures, second layer 520 is a top layer that encloses microfluidic channel 302f. However, it should be appreciated that in some embodiments, a microfluidic device may not include a top layer and a microfluidic channel may be open (e.g., to the external atmosphere), as the present disclosure is not limited in this regard. Microfluidic channel 302f includes first portion 241 and second portion 242. Both the first and second portions extend along the length of the microfluidic channel in the embodiment of FIGS. 11A and 11B, but in other embodiments a portion may extend in another direction and/or to a different extent. In the embodiment of FIGS. 11A and 11B, second portion 242 is spaced from first portion 241 in a second direction transverse to the first direction (i.e., transverse to the direction in which microfluidic channel 302f extends). In the embodiment of FIGS. 11A and 11B, second portion 242 is spaced from first portion 241 in a direction aligned with the X direction, as depicted in the figures.

In the embodiment of FIGS. 11A and 11B, a surface (e.g., a bottom surface) of microfluidic channel 302f is inclined in region 133 between first portion 241 and second portion 242. A height of first portion 241 is less than a height of second portion 242. The height of the inclined region 133 is variable such that the height of the inclined region transitions from approximately equal to the height of first portion 241 at a first end portion to approximately equal to the height of second portion 242 at a second end portion opposite the first end portion. The height of microfluidic channel 302f at different locations (e.g., the heights of first portion 241 and second portion 242) should be understood as extending in a third direction perpendicular to both the first and second directions. In FIGS. 11A and 11B, the height of the microfluidic channel extends along the Z direction.

Microfluidic device 100h may include reagent 250 disposed in microfluidic channel 302f. Specifically, in the depicted embodiment, reagent 250 may be disposed in second region 242. In some embodiments, reagent 250 may be disposed along at least a portion of a length of the second portion 242 of microfluidic channel 302f in the direction in which the microfluidic channel 302f extends (i.e., in the first direction aligned with the Y direction). The reagent may be disposed continuously along the microfluidic channel or in discrete groupings, as the disclosure is not so limited. For example, FIG. 11A shows three separate groupings of reagent 250, but it should be appreciated that greater or fewer numbers (and/or spacings) of groupings of reagent are contemplated.

Without wishing to be bound by theory, a microfluidic channel with different heights in different portions of the microfluidic channel may be associated with a capillary pressure gradient. As described above, the height of first portion 241 of microfluidic channel 302f is less than the height of second portion 242. As such, microfluidic channel 302f may be associated with a capillary pressure gradient in which the capillary pressure associated with first portion 241 may be greater than the capillary pressure associated with second portion 242. Accordingly, a fluid disposed within microfluidic channel 302f may be urged to flow from first portion 241 to second portion 242. That is, a fluid disposed within the microfluidic channel 302f may be urged to flow down the inclined region 133.

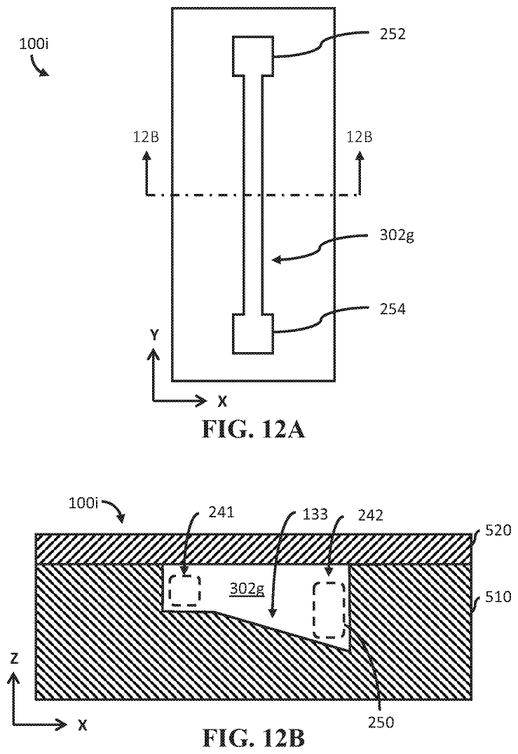

FIG. 12A depicts one embodiment of microfluidic device 100i, and FIG. 12B is a transverse cross-section of microfluidic device 100i of FIG. 12A. Microfluidic device 100i of FIGS. 12A and 12B is in many ways analogous to microfluidic device 100h of FIGS. 11A and 11B. Notably, microfluidic channel 302g of FIGS. 12A and 12B includes inlet 252 and outlet 254. Outlet 254 is spaced from inlet 252 in a first direction (i.e., in a direction in which microfluidic channel 302g extends, which is aligned with the Y direction in the figures). While the outlet is depicted as a single open reservoir, embodiments in which other microfluidic components are in fluid communication with the depicted outlet are also contemplated. Microfluidic device 100i comprises a plurality of layers, including first layer 510 and second layer 520. As elaborated on further below, the first layer may include a plurality of layers in some embodiments. Microfluidic channel 302g includes first portion 241 and second portion 242. Second portion 242 is spaced from first portion 241 in a second direction transverse to the first direction (i.e., transverse to the direction in which the microfluidic channel 302g extends). In the embodiment of FIGS. 12A and 12B, second portion 242 is spaced from first portion 241 in a direction aligned with the X direction, as depicted in the figures. A surface (e.g., a bottom surface) of microfluidic channel 302g is inclined in region 133 between first portion 241 and second portion 242. Microfluidic device 300c includes reagent 250 disposed in microfluidic channel 302g.

When fluid is introduced into inlet 252 of microfluidic channel 302g, the fluid may flow at least partially in the second direction before reaching outlet 254. Depending on certain parameters of the microfluidic channel and of the fluid, different flow patterns may be prescribed. As described above in reference to FIGS. 11A and 11B, a microfluidic channel with different heights in different portions of the microfluidic channel may be associated with a capillary pressure gradient. Because the height of first portion 241 of microfluidic channel 302g is less than the height of second portion 242, fluid introduced into inlet 252 of microfluidic channel 302g flows at least partially along a pressure gradient from first portion 241 toward second portion 242. That is, fluid introduced into inlet 252 of microfluidic channel 302g flows at least partially in a direction transverse to the length of microfluidic channel 302g. In this way, the fluid mixes with reagent 250 disposed in second portion 242 before exiting microfluidic channel 302g through the outlet 254. In some embodiments, mixing the fluid with the reagent may include reconstituting a dried reagent.

In some embodiments, a method may include introducing a fluid into a first portion of a microfluidic channel extending in a first direction. The first portion of the microfluidic channel may include an inlet of the microfluidic channel in some embodiments. The method may include flowing the fluid in a second direction transverse to the first direction from the first portion of the microfluidic channel to a second portion of the microfluidic channel. The first portion of the microfluidic channel and the second portion of the microfluidic channel may be at least partially coextensive along a length of the microfluidic channel. The method may include flowing the fluid out of the second portion of the microfluidic channel to an outlet of the microfluidic channel. The outlet of the microfluidic channel may be spaced from the first portion of the microfluidic channel in the first direction.

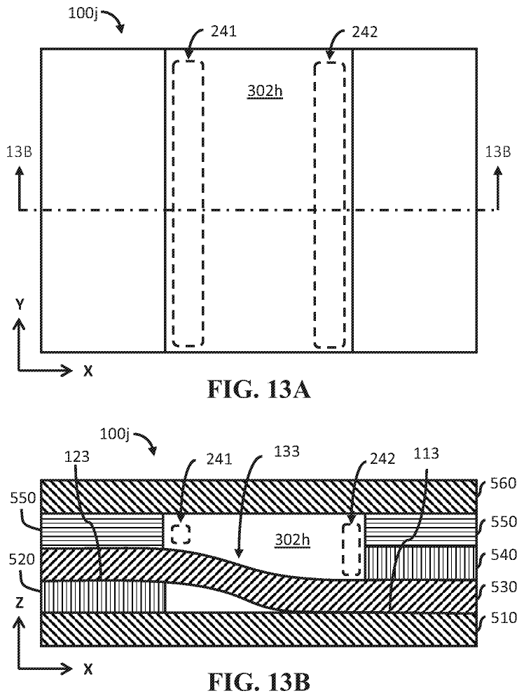

FIG. 13A depicts one embodiment of microfluidic device 100j, and FIG. 13B is a transverse cross-section of microfluidic device 100j of FIG. 13A. The depicted microfluidic device is similar to those described above relative to FIGS. 11A-11B, but is specifically constructed using patterned film layers that are stacked together to form the desired structure. In the figure, microfluidic device 100j includes microfluidic channel 302h, only a portion of which is shown in FIGS. 13A and 13B. The microfluidic channel extends in a first direction aligned with the Y direction shown in FIGS. 13A and 13B. Microfluidic device 100j comprises a plurality of layers, including first layer 510 (e.g., a substrate), second layer 520 (e.g., a first intermediate layer), third layer 530 (e.g., a second intermediate layer), fourth layer 540 (e.g., a third intermediate layer), fifth layer 550 (e.g., a fourth intermediate layer), and sixth layer 560 (e.g., a top layer). Second layer 520 is disposed on first layer 510. Third layer 530 is disposed on second layer 520 (at second location 123), and a portion of third layer 530 is directly disposed on first layer 510 (at first location 113). Fourth layer 540 is disposed on the third layer 530 (e.g., above first location 113). Fifth layer 550 is disposed on third layer 530 (e.g., above second location 123) and on fourth layer 540 (e.g., above first location 113). Sixth layer 560 is disposed on fifth layer 550.

Third layer 530 is inclined in region 133 between first location 113 and second location 123. Microfluidic device 100j of FIGS. 13A and 13B includes an inclined region 133. The inclined region 133 is between first portion 241 and second portion 242 of microfluidic channel 302h. A height of first portion 241 is less than a height of second portion 242. The height of the inclined region 133 is variable such that the height of the inclined region transitions from approximately equal to the height of first portion 241 at a first end portion to approximately equal to the height of second portion 242 at a second end portion opposite the first end portion. The height of the microfluidic channel 302h at different locations (e.g., the heights of first portion 241 and second portion 242) should be understood as extending in a third direction perpendicular to both the first and second directions. In FIGS. 13A and 13B, the height of the microfluidic channel extends along the Z direction. In contrast to the embodiment of FIGS. 10A and 10B, the inclined region 133 is inclined in a direction transverse to the direction in which the microfluidic channel 302h extends. That is, the microfluidic channel 302h extends in the Y direction, and the inclined region 133 is inclined in the X direction.

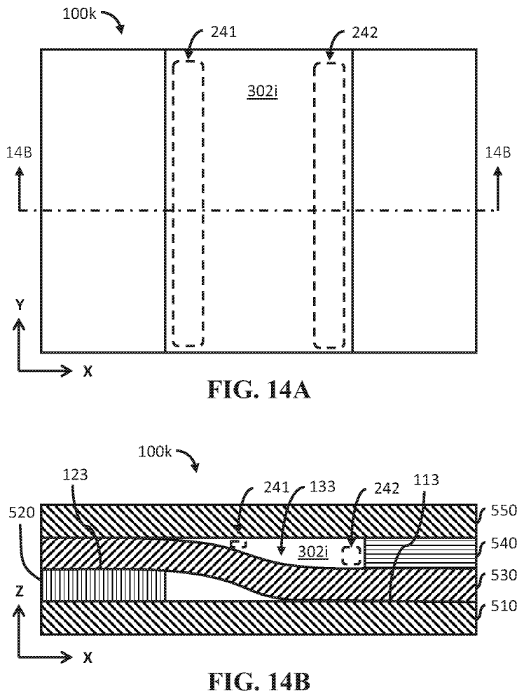

FIG. 14A depicts one embodiment of microfluidic device 100k, and FIG. 14B is a transverse cross-section of microfluidic device 100k of FIG. 14A. The depicted microfluidic device is similar to the microfluidic device 100j described above relative to FIGS. 13A-13B, but includes fewer layers. In the figure, the microfluidic device 100k includes microfluidic channel 302i, only a portion of which is shown in FIGS. 14A and 14B. The microfluidic channel extends in a first direction aligned with the Y direction shown in FIGS. 14A and 14B. Microfluidic device 302i comprises a plurality of layers, including first layer 510 (e.g., a substrate), second layer 520 (e.g., a first intermediate layer), third layer 530 (e.g., a second intermediate layer), fourth layer 540 (e.g., a third intermediate layer), and fifth layer 550 (e.g., a top layer). Second layer 520 is disposed on first layer 510. Third layer 530 is disposed on second layer 520 (at second location 123), and a portion of third layer 530 is directly disposed on first layer 510 (at first location 113). Fourth layer 540 is disposed on third layer 530 (e.g., above first location 113). Fifth layer 550 is disposed on third layer 530 (e.g., above second location 123) and on fourth layer 540 (e.g., above first location 113).

Third layer 530 is inclined in region 133 between first location 113 and second location 123. Microfluidic device 100k of FIGS. 14A and 14B includes an inclined region 133. The inclined region 133 is between first portion 241 and second portion 242 of microfluidic channel 100k. A height of first portion 241 is less than a height of second portion 242. The height of the inclined region 133 is variable such that the height of the inclined region transitions from approximately equal to the height of first portion 241 at a first end portion to approximately equal to the height of second portion 242 at a second end portion opposite the first end portion. The height of microfluidic channel 302i at different locations (e.g., the heights of first portion 241 and second portion 242) should be understood as extending in a third direction perpendicular to both the first and second directions. In FIGS. 14A and 14B, the height of the microfluidic channel extends along the Z direction. In contrast to the embodiment of FIGS. 10A and 10B, the inclined region 133 is inclined in a direction transverse to the direction in which the microfluidic channel 302i extends. That is, the microfluidic channel 302i extends in the Y direction, and the inclined region 133 is inclined in the X direction.

It should be appreciated that different combinations and/or arrangements of layers may be appropriate in different embodiments of a microfluidic device. In some embodiments, each layer may be associated with a separate film of material. In some embodiments, a single film of material may be associated with multiple layers. For example, a planar film of material may be partially cut to form a flap that is subsequently tucked underneath or disposed on top of another portion of the same film of material, thereby forming an inclined surface. In this way, a single planar film of material may be modified to form multiple separate layers of a microfluidic device.

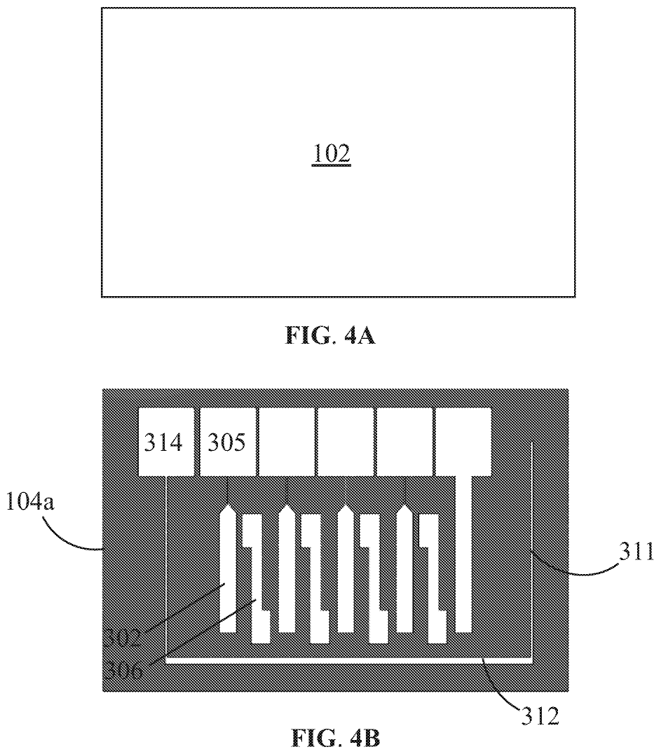

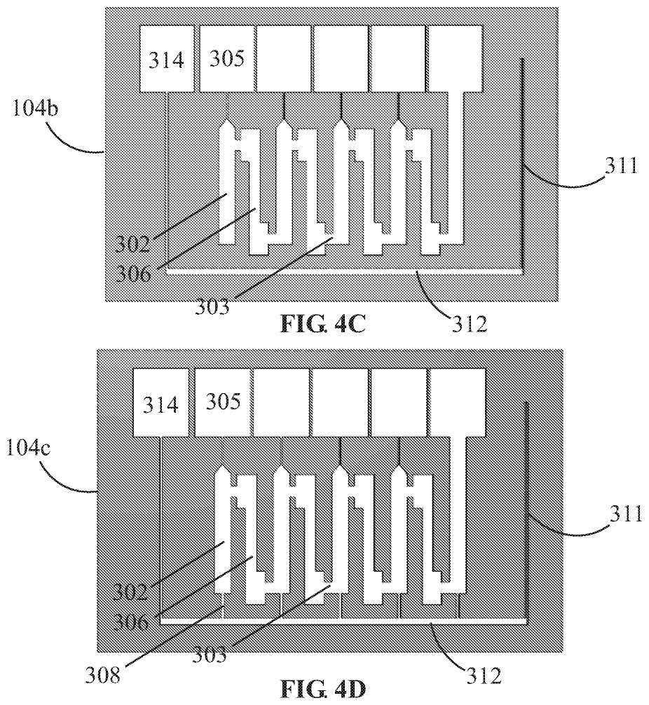

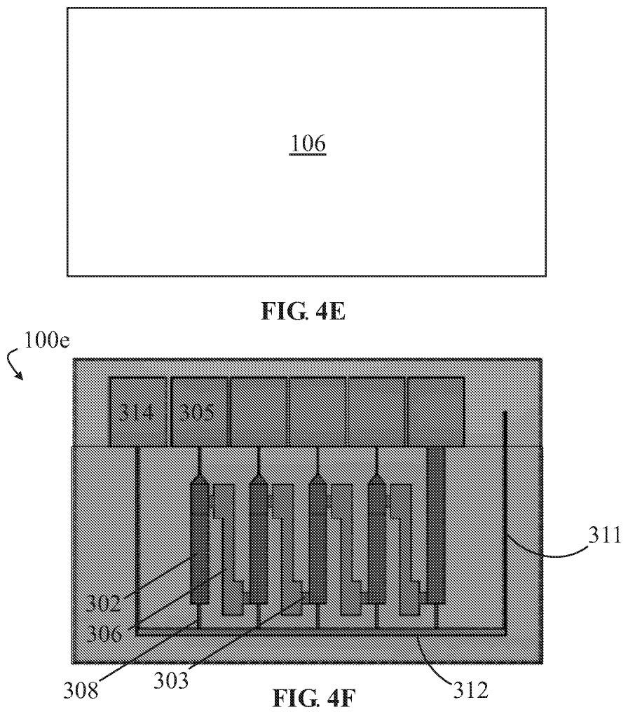

FIGS. 4A-4E show, according to some embodiments, top-view schematic diagrams of hydrophilic substrate 102, hydrophobic first intermediate layer 104a, hydrophilic second intermediate layer 104b, third intermediate layer 104c (the top and bottom surfaces of this layer are not exposed to liquids, and thus, the hydrophobicity is not as relevant), and a hydrophobic top layer 106, respectively. FIG. 4F shows, according to some embodiments, a top-view schematic diagram of microfluidic device 100e containing the layers shown in FIGS. 4A-4E in a stacked configuration. In the depicted embodiment, plurality of inlet reservoirs 305; a corresponding plurality of liquid channels 302 fluidly connected to plurality of inlet reservoirs 305; a corresponding plurality of pneumatic channels 306; inlet channel 311; and primary channel 312 fluidly connected to inlet channel 311 are formed in each of the intermediate layers. Accordingly, when the intermediate layers are stacked together, the reservoirs, the liquid channels, the pneumatic channels, the inlet channel, and the primary channel are formed between opposing surfaces of the substrate and top layer with the side surfaces extending between the substrate and top layer formed by the surfaces of the patterned portions of the intermediate layers.

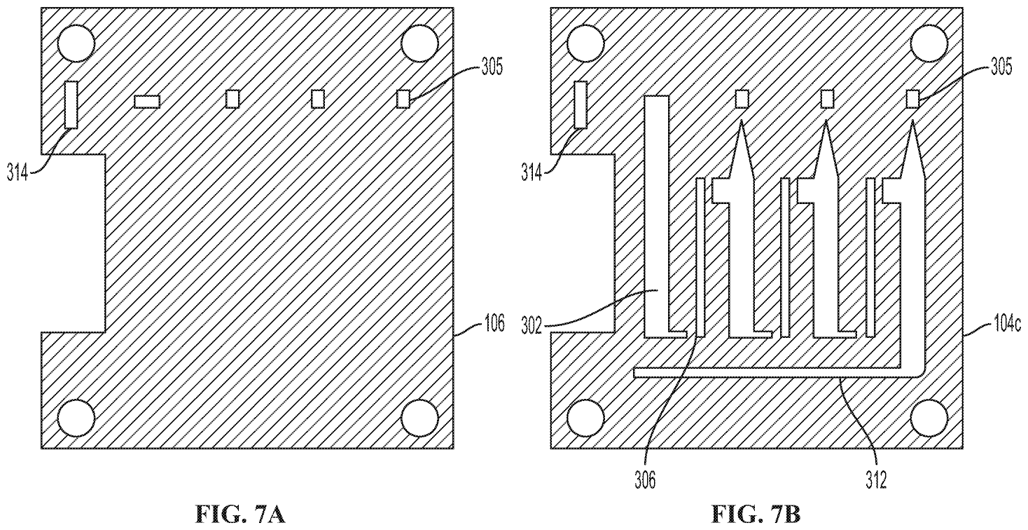

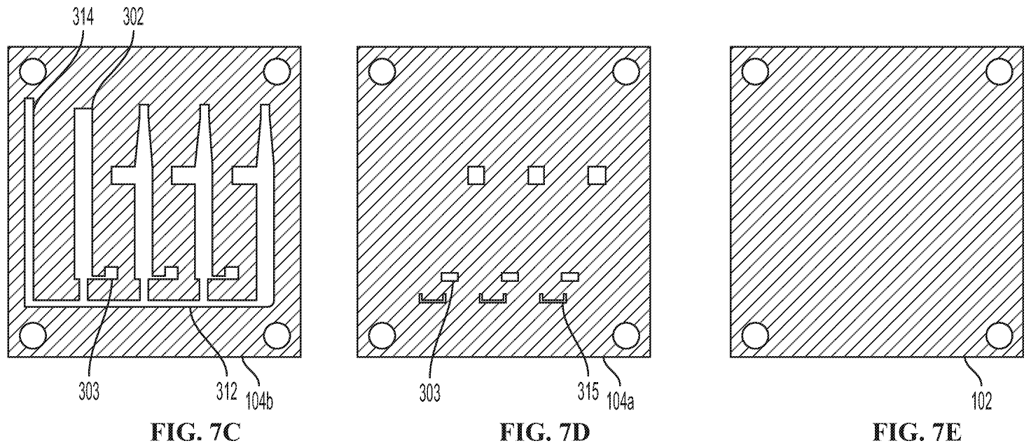

FIGS. 7A-7E show an alternate embodiment of substrate 102, first intermediate layer 104a, second intermediate layer 104b, third intermediate layer 104c, and top layer 106, in which a plurality of fluidly connected microfluidic components (e.g., channel 302, inlet reservoirs 305, pneumatic channel 306, primary channel 312, outlet 314, and stop valve 303) are defined by one or more of the layers of the device. FIG. 7A shows, according to some embodiments, top layer 106 defining inlet reservoirs 305 and outlet 314. FIG. 7B shows, in some embodiments, third intermediate layer 104c defining microfluidic channels 302, pneumatic channels 306, primary channel 312, inlet reservoirs 305, and outlet 314. As shown in FIG. 7B, a portion of third intermediate layer 104c exposed to an interior of a portion of microfluidic channel 302 (e.g., in second intermediate layer 104b) has a first transverse dimension at an upstream location and a second transverse dimension at a downstream location, wherein the first transverse dimension is smaller than the second transverse dimension. FIG. 7C shows, according to some embodiments, second intermediate layer 104b defining microfluidic channels 302, stop valves 303, primary channel 312, and outlet 314. FIG. 7D shows, according to some embodiments, first intermediate layer 104a defining stop valves 303. FIG. 7E shows, in some embodiments, substrate 102.

In some embodiments, stop valves 303 are disposed between each adjacent liquid channel and pneumatic channel and may correspond to channels formed in the intermediate layers (e.g., the second and third intermediate layers). Accordingly, liquid present in one liquid channel is pinned at the interface with the stop valves formed in the intermediate layers due to the unpatterned hydrophobic surface of the first intermediate layer being exposed to the channel forming the stop valve or due to hydrophobic side walls formed by cutting through one or more hydrophilic surfaces of the layer, and thus, preventing liquid from entering the pneumatic channel.

Plurality of trigger valves 308 in fluid communication with each liquid channel may provide fluid communication between the associated liquid channel and primary channel 312. In the depicted embodiment, the trigger valves are formed in the third intermediate layer. Thus, the hydrophilic surface of the second intermediate layer exposed to the interior of the trigger valves may draw liquid to the interface between the trigger valves and the primary channel. Accordingly, liquid flowing through the primary channel may trigger flow from the reservoirs to the primary channel through the trigger valves as each trigger valve is triggered in sequence due to the flow of liquid passing through the primary channel. The combined flow of liquids may then flow into outlet 314 formed in each of the intermediate layers. The liquid flow can be triggered and/or stopped by forming hydrophobic barrier 315 at the end of one or more channel. In some embodiments, hydrophobic barrier 315 may be engraved and/or cut to stop liquid flow at the end of the one or more channels and trigger liquid flow from the one or more channels by flowing a liquid in the primary channel.

The microfluidic device may be used for any of a variety of suitable applications. In some embodiments, for example, the microfluidic device is used to perform affinity-based electrochemical detection. Accordingly, in some embodiments, the microfluidic device may comprise or otherwise be associated with an electrochemical detection sensor. The electrochemical detection may be performed, in some embodiments, by delivering different liquid samples and reagents sequentially from the reservoirs defined by the one or more intermediate layers. In certain embodiments, an electrochemical detection sensor (e.g., with conjugated antibodies) is placed proximate to an outlet channel or reaction chamber. Liquid samples and/or reagent may be added to an inlet of each reservoir, in some embodiments. The reservoirs may contain any of a variety of suitable samples and/or reagents. Examples include, but are not limited to, a blood sample, a plasma sample, a detection antibody, a wash buffer (e.g., phosphate-buffered saline with Tween® (PBST)), horseradish peroxidase (HRP)-conjugated streptavidin, and/or tetramethylbenzidine (TMB). In certain embodiments, stop valves at the end of the reservoirs will stop the flow to aliquot the liquid volume needed for electrochemical detection. Liquid flow from the reservoirs is triggered, in some embodiments, by adding a fluid (e.g., buffer) through the main channel of the microfluidic device (see, for example, primary channel 312 in FIG. 4F). The microfluidic device may drain the liquid samples and/or reagents from the reservoirs sequentially and/or simultaneously to initiate mixing and/or deliver the samples and/or reagents to the outlet, electrochemical detection sensor, other sensor, or any other appropriate component as the disclosure is not limited in this fashion.

### Example 1

The following example describes the fabrication and operation of a microfluidic device.

A capillary microfluidic device was fabricated from layers of single- and double-sided adhesive tapes and thin films without any adhesive sides, similar to the system shown in FIGS. 4A-4F. The first layer was a hydrophilic layer that was used to generate the capillary pressure used to induce flow at the bottom of the channels. The first layer was made from a hydrophilic glass slide (75×50×1 mm).

The second layer was designed to define the channels and the stop valves. The second layer was made from a 100 μm thick hydrophobic adhesive tape (e.g., 3M™ Microfluidic Diagnostic Tape 9795R or Single Sided Clear Delayed Tack Adhesive Tape). The stop valve functional section was located on the non-adhesive side of this tape.

The third layer was designed to allow liquid to flow above the stop valves. The side walls of the hydraulic channels were formed from the third layer. The third layer was mainly used to define the thickness of the channels and the volumes stored inside. The third layer was made from a 100 μm thick hydrophobic adhesive tape (e.g., 3M™ Microfluidic Diagnostic Tape 9795R). Thicker films could also be used if desired to increase the volume of features formed in this layer. The top side of the third layer was hydrophobic to avoid any liquid flow through the pneumatic air channels on the layer above it.

The fourth layer was used to define the pneumatic channels. The fourth layer was made from a 100 μm thick hydrophobic adhesive tape (e.g. 3M™ Microfluidic Diagnostic Tape 9795R). The fourth layer formed the side walls of the pneumatic air channels. The bottom part of the fourth layer was used to cover parts of the hydraulic channels. The bottom side of the fourth layer was hydrophobic to stop the liquid at the stop valves. The water contact angle of the adhesive bottom side of the fourth layer was 91 degrees.

The last layer was a hydrophobic layer used to cover the channels and the pneumatic layer. The last layer was made from a 100 μm thick hydrophobic adhesive tape (e.g., 3M™ Microfluidic Diagnostic Tape 9795R). The bottom part of the last layer was hydrophobic to avoid any liquid flow through the pneumatic air channels in the layer below it. All the layers were assembled by applying pressure manually on the adhesive tapes which included pressure sensitive adhesives.

The assembled microfluidic device is capable of being operated to perform affinity-based electrochemical detection on the device. Specifically, the manufactured device was capable of delivering different liquid samples and reagents sequentially to a biosensor as follows. First, an electrochemical detection sensor with conjugated antibodies on the surface was placed in fluid communication with an outlet channel of the device. Liquid samples and reagents were added at the inlets of each reservoir. The stop valves at the end of these reservoirs were configured to stop the flow to aliquot the liquid volume used for the next operations. The flow was triggered from these reservoirs by adding buffer through the inlet and primary channel located at the bottom of the device. Liquids were stored in the reservoirs according to the following sequence:
Reservoir 1: Blood sample
Reservoir 2: Detection antibody
Reservoir 3: Wash buffer: phosphate buffered saline (PBST)
Reservoir 4: Horseradish peroxidase (HRP)-conjugated streptavidin
Reservoir 5: Wash buffer: PBST
Reservoir 6: Tetramethylbenzidine (TMB)
Reservoir 7: Wash buffer: PBST.

The device started running by draining the first and the second reservoirs simultaneously to mix the blood sample with the detection antibody. The mixture then flowed on the surface of the sensor. After draining the detection antibody in the second reservoir, the air valve opened and allowed the wash buffer in the third reservoir to drain and flow over the sensor. After the wash buffer was drained, the flow of the HRP-conjugated streptavidin was initiated by opening the air valve. The next air valve opened and another washing step was performed followed by delivering TMB on the sensor. After draining the TMB, the air valve for the last reservoir opened and allowed for the last washing step for the sensor.

### Example 2

The following example describes the operation of a microfluidic device for the electrochemical detection of COVID-19 antibodies.

A microfluidic device was fabricated according to Example 1. Electrochemical detection sensor 900 was placed in fluid communication with an outlet channel of microfluidic device 100f, as shown in FIG. 15. The electrochemical detection sensor included a first electrode with nucleocapsid (NC) protein spotted on the electrode and a second control electrode. Liquid samples and reagents were added at the inlets of each reservoir. The stop valves at the end of these reservoirs were configured to stop the flow to aliquot the liquid volume used for the next operations. The flow was triggered from these reservoirs by adding buffer through the inlet and primary channel located at the bottom of the chip. Liquids were stored in the reservoirs according to the following sequence:
Reservoir 1: Plasma sample
Reservoir 2: Detection antibody with HRP
Reservoir 3: Tetramethylbenzidine (TMB); and
Reservoir 4: Wash buffer: PBS

The device started running by delivering the sample and the reagents sequentially to the electrochemical sensor, followed by a final washing step with PBS buffer.

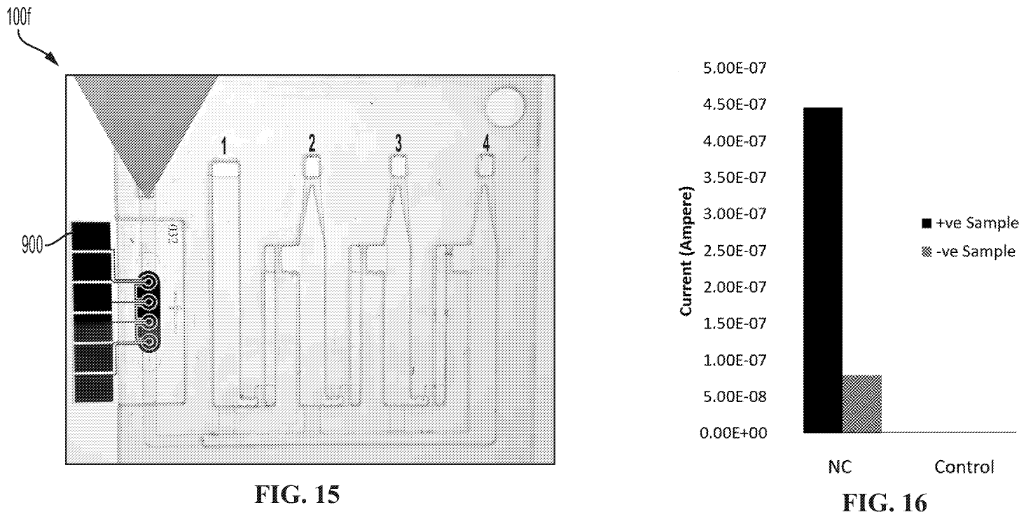

The working electrodes in the electrochemical sensor were spotted with NC protein that was used to capture the antibody in the plasma sample. The control electrodes were blocked with bovine serum albumin (BSA). The positive plasma sample had COVID-19 antibodies that bind to the NC protein spotted on the surface of the electrode. The enzymatic amplification steps were performed by flowing the detection antibody with HRP then TMB. FIG. 16 shows the significant difference in the signal between the positive and the negative sample where there is no signal for the control electrodes that are blocked with bovine serum albumin (BSA).

### Example 3

The following example describes a microfluidic device comprising a microfluidic channel having a gap or area of increased hydrophobicity in between two separated portions of the channel to separately pin one or more liquids in desired portions of the channel.

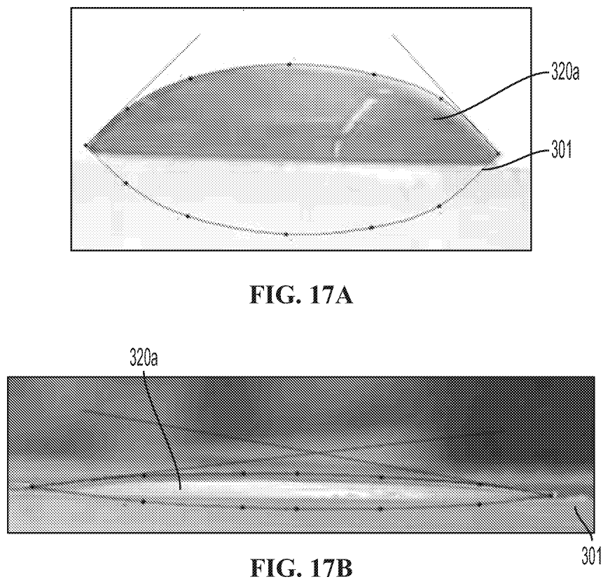

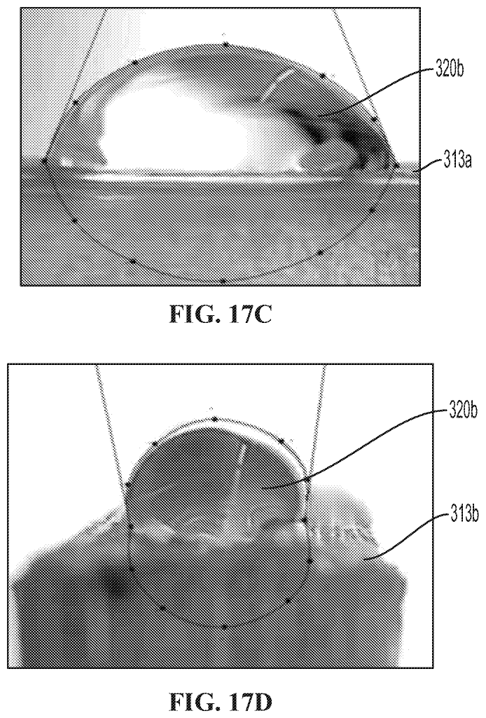

FIGS. 17A-17B show embodiments illustrating the effect of surface treatment on hydrophilicity or hydrophobicity, in accordance with some embodiments. FIG. 17A is a side-view embodiment that shows volume 320a of 0.1% PBST on a surface of 3D-printed resin 301 before surface treatment, with a contact angle of 46 degrees. FIG. 17B is a side-view embodiment that shows volume 320a of 0.1% PBST on the surface of 3D-printed resin 301 after plasma treatment, with a contact angle of 8 degrees. The results in FIGS. 17A and 17B show that the plasma treatment can modify the resin surface to become more hydrophilic and less hydrophobic.

FIGS. 17C-17D show embodiments illustrating the effect of different surfaces of films on hydrophilicity or hydrophobicity, in accordance with certain embodiments. FIG. 17C is a side-view embodiment of a flat surface of a plurality of stacked double-sided tapes with volume 320b of 0.1% PBST on the flat upper surface 313a of the uppermost layer of the double-sided tape, with a contact angle of 69 degrees. FIG. 17D is a side-view embodiment that shows volume 320b of 0.1% PBST on the side surfaces 313b of the plurality of stacked double-sided tapes, with a contact angle of 99 degrees. The results in FIGS. 17C and 17D show that the stacked tapes, which may function as layers of a microfluidic device, may exhibit different hydrophobicities on different surfaces of the plurality of stacked double-sided tapes, and in this embodiment a larger hydrophobicity on the side surfaces of the depicted double-sided tapes, which may be used to fabricate one or more microfluidic channels of a microfluidic device, as explained in further detail below. However, side surfaces of a plurality of stacked double-sided tapes, or other films, that are less hydrophobic than an upper and/or lower surface of a film are also contemplated.

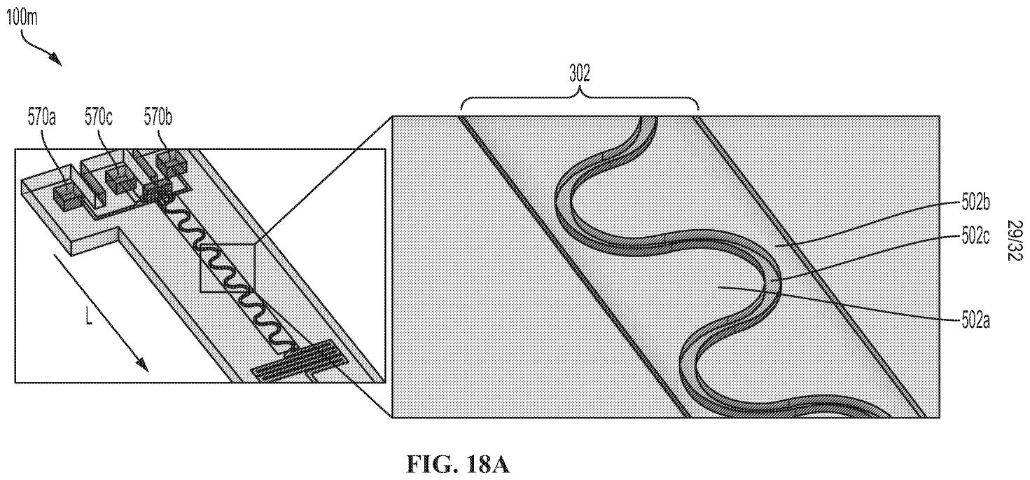

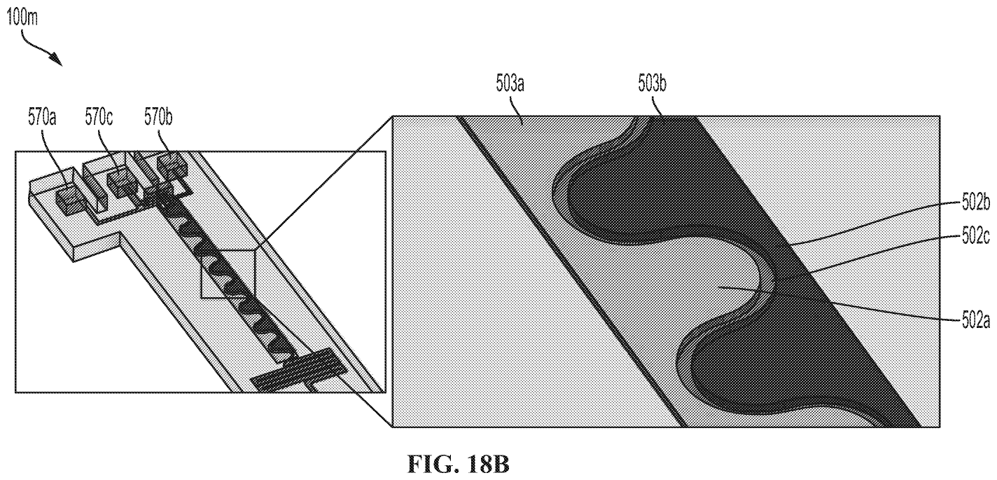

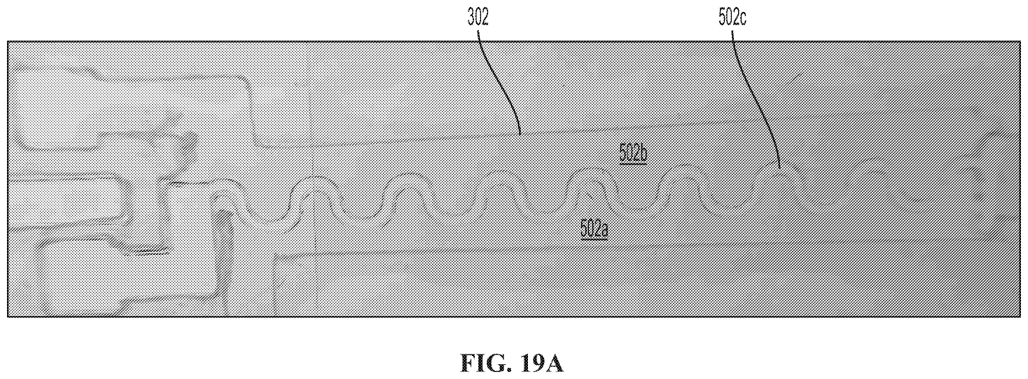

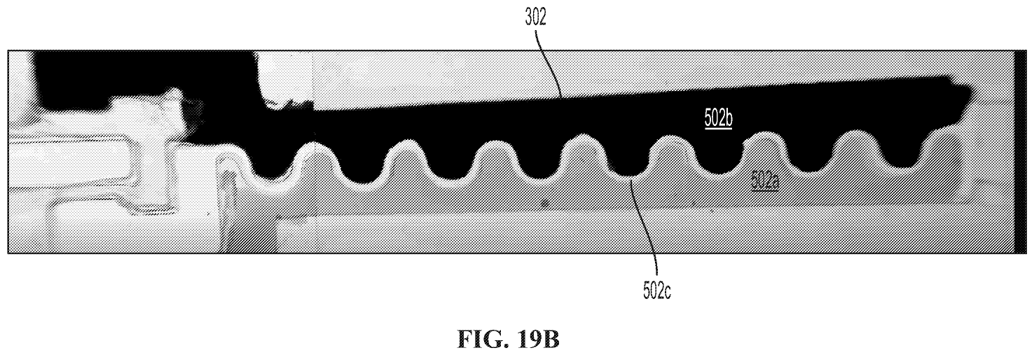

The plurality of stacked double-sided tapes may be used to fabricate one or more passive components (e.g., microfluidic channels) of a microfluidic device. FIG. 18A shows a schematic diagram of microfluidic device 100m, in accordance with some embodiments. Device 100m has channel 302 that may be constructed similarly to channel 302d as shown in FIG. 9A (e.g., wherein channel 302d consists of a recessed channel and a primary channel in the space enclosed between substrate 102, first intermediate layer 104a, second intermediate layer 104b, and top layer 106, which may each be a layer of a film such as a double-sided tape). In FIG. 18A, channel 302 comprises middle portion 502c disposed between first portion 502a and second portion 502b, all of which constitute a primary channel. Middle portion 502c may be a recessed channel that extends below the first and second portions of the primary channel. As shown in FIG. 18A, each of middle portion 502c, first portion 502a and second portion 502b extend along a longitudinal direction L of the channel. In the example shown, portions 502a, 502b, and 502c extend along a non-linear path, which may be a meandering or a serpentine path, although it should be appreciated that any suitable linear or non-linear path may be used to transport substances along a length of the channel as aspects of the present disclosure are not so limited. Without wishing to be bound by theory, a non-linear path such as the depicted serpentine path may help to facilitate intermixing of the substances included in the isolated first and second portions of the primary channel. FIG. 19A shows an embodiments of the schematic diagram shown in FIG. 18A, wherein channel 302 has been formed by a plurality of films.

Referring to FIG. 18B, first substance 503a is disposed in first portion 502a of channel 302. In an exemplary process, microfluidic pump 570a may be used to provide first substance 503a, although any other suitable means such as capillary pressure induced flow, paper pumps, or valves may be used. Microfluidic pump 570b may be used to provide second substance 503b in second portion 502b of channel 302, although any other suitable means such as capillary pressure induced flow, paper pumps, or valves may be used. When middle portion 502c is empty, the two substances 503a and 503b remain separated and cannot mix with each other. FIG. 19B shows an embodiments of the schematic diagram shown in FIG. 18B, wherein channel 302 has been formed by a plurality of films.

Subsequently, a liquid (not shown) may be provided in middle portion 502c. The liquid may be provided by, for example microfluidic pump 570c, although other ways of providing a liquid into the various portions of the channel may be used including, for example, capillary pressure induced flow as the disclosure is not limited to how the liquids or other substances are introduced to the various portions of the channel. In either case, upon flowing the liquid through middle portion 502c, the liquid in the middle channel may merge with one or both of the first and second substances on both sides of the middle channel. This may then permit intermixing of the substances disposed in the first and second portions of the primary channel.

In some embodiments, the first substance and the second substance are both liquids. When a third liquid is added in the middle portion 502c, it flows in the defined path that was acting as a stop valve for the two liquids lying on the sides in the first and second portions 502a and 502b. The volume of liquids on the sides can be varied in different ratios to enable having a desired dilution ratio. In some embodiments, the entire channel 302 can act as a reservoir and be drained as a complete one liquid block once all the liquids become merged and connected inside the reservoir.

While several embodiments have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A microfluidic device, comprising:
a substrate configured to facilitate fluid transport;
one or more intermediate layers disposed on the substrate, wherein the one or more intermediate layers are configured to at least partially define a plurality of fluidly connected microfluidic components; and
a top layer disposed on the one or more intermediate layers.

2. The microfluidic device of claim 1, wherein the substrate, one or more intermediate layers, and the top layer each comprise an adhesive.

3. The microfluidic device of claim 1, wherein the substrate, one or more intermediate layers, and the top layer are bonded together without an adhesive.

4. The microfluidic device of any one of claims 1-3, wherein a side of the substrate facing the one or more intermediate layers has a water contact angle of greater than or equal to 10 degrees and less than or equal to 180 degrees.

5. The microfluidic device of any one of claims 1-4, wherein the substrate is configured to facilitate fluid flow through at least a portion of the plurality of fluidly connected microfluidic components.

6. The microfluidic device of any one of claims 1-5, wherein the substrate comprises a hydrophilic material.

7. The microfluidic device of any one of claims 1-6, wherein the substrate, one or more intermediate layers, and/or the top layer comprise a polymer.

8. The microfluidic device of any one of claims 1-7, wherein the polymer comprises polypropylene, polydimethylsiloxane, a cyclic olefin copolymer, poly(methyl methacrylate), and/or a photopolymer used in 3D-printing.

9. The microfluidic device of any one of claims 1-7, wherein the substrate, one or more intermediate layers, and/or the top layer comprise polyester.

10. The microfluidic device of any one of claims 1-6, wherein the substrate comprise glass.

11. The microfluidic device of any one of claims 1-10, wherein the substrate, one or more intermediate layers, and/or the top layer each have a thickness greater than or equal to 1 micrometer and less than or equal to 2 millimeters.

12. The microfluidic device of any one of claims 1-10, wherein the substrate, one or more intermediate layers, and/or the top layer each have a thickness greater than or equal to 50 micrometers and less than or equal to 150 micrometers.

13. The microfluidic device of any one of claims 1-12, wherein the top layer comprises a hydrophobic material.

14. The microfluidic device of any one of claims 1-13, wherein at least one of the one or more intermediate layers comprises a hydrophobic material.

15. The microfluidic device of any one of claims 1-13, wherein at least one of the one or more intermediate layers comprises a hydrophilic material.

16. The microfluidic device of any one of claims 1-15, wherein the one or more intermediate layers comprise a plurality of intermediate layers.

17. The microfluidic device of claim 16, wherein at least one of the plurality of intermediate layers has a hydrophobicity that is different from at least one other of the plurality of intermediate layers.

18. The microfluidic device of any one of claims 1-17, wherein the plurality of fluidly connected microfluidic components includes a plurality of microfluidic channels, reservoirs, and/or valves.

19. The microfluidic device of any one of claims 1-18, wherein a hydrophobicity of the top layer and/or the one or more intermediate layers is different from a hydrophobicity of the substrate.

20. The microfluidic device of claim 18, wherein a bottom surface of at least a portion of at least one microfluidic channel has a first hydrophilicity or hydrophobicity and one or more side walls of at least a portion of the at least one microfluidic channel has a second hydrophilicity or hydrophobicity that is greater than the first hydrophilicity or hydrophobicity.

21. The microfluidic device of claim 20, wherein the bottom surface of at least a portion of the at least one microfluidic channel is defined by the substrate and the one or more side walls of at least a portion of the at least one microfluidic channel is defined by one or more intermediate layers.

22. The microfluidic device of claim 20, wherein the bottom surface of at least a portion of the at least one microfluidic channel is defined by a first intermediate layer and the one or more side walls of at least a portion of the at least one microfluidic channel is defined by at least a second intermediate layer.

23. The microfluidic device of any one of claims 20-22, wherein the portion of the at least one microfluidic channel is configured to function as a stop valve.

24. The microfluidic device of claim 18, wherein a first microfluidic channel intersects with a second microfluidic channel.

25. The microfluidic device of claim 24, wherein the first microfluidic channel and the second microfluidic channel are defined by one intermediate layer.

26. The microfluidic device of any one of claims 24-25, wherein an interface between the first microfluidic channel and the second microfluidic channel is patterned such that the interface is configured to function as a stop valve.

27. The microfluidic device of claim 26, wherein the interface between the first microfluidic channel and the second microfluidic channel comprises a triangular geometry.

28. The microfluidic device of any one of claims 26-27, wherein the interface between the first microfluidic channel and the second microfluidic channel has a hydrophobicity or hydrophilicity that is different than the hydrophobicity or hydrophilicity of the first microfluidic channel.

29. The microfluidic device of any one of claims 26-28, wherein the interface between the first microfluidic channel and the second microfluidic channel has a hydrophobicity or hydrophilicity that is different than the hydrophobicity or hydrophilicity of the second microfluidic channel.

30. The microfluidic device of claim 18, wherein a portion of least one microfluidic channel is fluidly connected to and proximate a reservoir.

31. The microfluidic device of claim 30, wherein the portion of the at least one microfluidic channel and the reservoir are defined by at least a first intermediate layer and a second intermediate layer, wherein the second intermediate layer is a hydrophobic layer that is more hydrophobic than the first intermediate layer.

32. The microfluidic device of claim 31, wherein a portion of the hydrophobic layer exposed to an interior of the portion of the at least one microfluidic channel has a first transverse dimension at an upstream location and a second transverse dimension at a downstream location, wherein the first transverse dimension is smaller than the second transverse dimension such that the portion of the at least one microfluidic channel is configured to act as a retention valve

33. The microfluidic device of claim 32, wherein the first transverse dimension and the second transverse dimension comprise a triangular dimension.

34. The microfluidic device of any one of claims 1-33, further comprising an electrochemical detection sensor.

35. A method of manufacturing a microfluidic device comprising:
applying one or more intermediate layers to a substrate to define a plurality of fluidly connected microfluidic components; and
applying a top layer to a topmost one of the one or more intermediate layers.

36. The method of claim 35, further comprising bonding the substrate, the one or more intermediate layers, and the top layer to each other using an adhesive.

37. The method of any one of claims 35-36, further comprising bonding the substrate, the one or more intermediate layers, and the top layer to each other without an adhesive.

38. The method of any one of claims 35-37, wherein the substrate comprises a hydrophilic material.

39. The method of any one of claims 35-38, wherein the substrate, one or more intermediate layers, and/or the top layer comprise polyester.

40. The method of any one of claims 35-38, wherein the substrate comprises glass.

41. The method of any one of claims 35-40, wherein the top layer comprises a hydrophobic material.

42. The method of any one of claims 35-41, wherein at least one of the one or more intermediate layers comprises a hydrophobic material.

43. The method of any one of claims 35-41, wherein at least one of the one or more intermediate layers comprises a hydrophilic material.

44. The method of any one of claims 35-43, wherein the one or more intermediate layers comprise a plurality of intermediate layers.

45. The method of claim 44, wherein at least one of the plurality of intermediate layers has a hydrophobicity that is different from at least one other of the plurality of intermediate layers.

46. The method of any one of claims 35-45, wherein the plurality of fluidly connected microfluidic components includes a plurality of microfluidic channels, reservoirs, and/or valves.

47. The method of any one of claims 35-46, wherein a hydrophobicity of the top layer and/or the one or more intermediate layers is different from a hydrophobicity of the substrate.

48. A microfluidic device, comprising
a plurality of microfluidic channels,
wherein at least a portion of at least one microfluidic channel has a transverse cross-section that is orthogonal to a direction of flow through the at least one microfluidic channel, and wherein a first portion of the transverse cross-section has a first hydrophobicity or hydrophilicity and a second portion of the transverse cross-section has a second hydrophobicity or hydrophilicity that is different than the first hydrophobicity or hydrophilicity.

49. The microfluidic device of claim 48, wherein the plurality of microfluidic channels is defined by one or more intermediate layers.

50. The microfluidic device of claim 49, wherein the one or more intermediate layers are disposed on a substrate.

51. The microfluidic device of any one of claims 49-50, wherein a top layer is disposed on the one or more intermediate layers.

52. The microfluidic device of any one of claims 48-51, wherein the portion of the at least one microfluidic channel is configured to function as a stop valve.

53. The microfluidic device of any one of claims 48-51, wherein the portion of the at least one microfluidic channel is configured to function as a trigger valve.

54. A microfluidic device, comprising:
a substrate configured to facilitate fluid transport;
one or more intermediate layers disposed on the substrate, wherein the one or more intermediate layers are configured to at least partially define a plurality of microfluidic channels; and
a top layer disposed on the one or more intermediate layers,
wherein a bottom surface of at least a portion of at least one microfluidic channel has a first hydrophilicity or hydrophobicity and one or more side walls of at least a portion of the at least one microfluidic channel has a second hydrophilicity or hydrophobicity that is greater than the first hydrophilicity or hydrophobicity.

55. The microfluidic device of claim 54, wherein the bottom surface of at least a portion of the at least one microfluidic channel is defined by the substrate and the one or more side walls of at least a portion of the at least one microfluidic channel is defined by one or more intermediate layers.

56. The microfluidic device of claim 55, wherein the one or more side walls of at least a portion of the at least one microfluidic channel is defined by one intermediate layer.

57. The microfluidic device of claim 55, wherein the one or more side walls of at least a portion of the at least one microfluidic channel is defined by more than one intermediate layer.

58. The microfluidic device of claim 54, wherein the bottom surface of at least a portion of the at least one microfluidic channel is defined by a first intermediate layer and the one or more side walls of at least a portion of the at least one microfluidic channel is defined by at least a second intermediate layer.

59. The microfluidic device of claim 58, wherein the one or more side walls of at least a portion of the at least one microfluidic channel is defined by a second intermediate layer.

60. The microfluidic device of claim 58, wherein the one or more side walls of at least a portion of the at least one microfluidic channel is defined by more than one intermediate layer.

61. The microfluidic device of any one of claims 54-60, wherein the portion of the at least one microfluidic channel is configured to function as a stop valve.

62. A microfluidic device, comprising:
a substrate configured to facilitate fluid transport;
an intermediate layer disposed on the substrate, wherein the intermediate layer is configured to at least partially define a first microfluidic channel and a second microfluidic that intersect with each other; and
a top layer disposed on the one or more intermediate layers,
wherein an interface between the first microfluidic channel and the second microfluidic channel is patterned such that the interface is configured to function as a stop valve.

63. The microfluidic device of claim 62, wherein the interface between the first microfluidic channel and the second microfluidic channel comprises a triangular pattern.

64. The microfluidic device of any one of claims 62-63, wherein the interface between the first microfluidic channel and the second microfluidic channel is laser etched and/or engraved.

65. The microfluidic device of any one of claims 62-64, wherein the interface between the first microfluidic channel and the second microfluidic channel has a hydrophobicity or hydrophilicity that is different than the hydrophobicity or hydrophilicity of the first microfluidic channel.

66. The microfluidic device of any one of claims 62-65, wherein the interface between the first microfluidic channel and the second microfluidic channel has a hydrophobicity or hydrophilicity that is different than the hydrophobicity or hydrophilicity of the second microfluidic channel.

67. A microfluidic device, comprising:
a substrate configured to facilitate fluid transport;
one or more intermediate layers disposed on the substrate, wherein the one or more intermediate layers are configured to at least partially define a microfluidic channel; and
a hydrophobic layer disposed on the one or more intermediate layers, wherein the hydrophobic layer is more hydrophobic than the intermediate layers, and
wherein a portion of the hydrophobic layer exposed to an interior of a portion of the microfluidic channel has a first transverse dimension at an upstream location and a second transverse dimension at a downstream location, wherein the first transverse dimension is smaller than the second transverse dimension such that the portion of the microfluidic channel is configured to act as a retention valve.

68. The microfluidic device of claim 67, wherein the first transverse dimension and the second transverse dimension comprise a triangular dimension.

69. A microfluidic device, comprising:
a channel including:
a first portion extending along a length of the channel;
a second portion extending along the length of the channel; and
a third portion extending along the length of the channel, wherein the third portion is disposed between the first portion of the channel and the second portion of the channel, and wherein the third portion is configured to isolate the first portion of the channel from the second portion of the channel until a liquid is flowed through the third portion of the channel.

70. The microfluidic device of claim 69, wherein at least one surface of the third portion of the channel is more hydrophobic than adjacent surfaces of the first portion of the channel and the second portion of the channel.

71. The microfluidic device of claim 69, wherein the third portion of the channel is a recessed portion of the channel extending along the length of the channel between the first portion of the channel and the second portion of the channel.

72. The microfluidic device of claim 71, wherein:
the recessed portion of the channel comprises a bottom surface and a side wall above the bottom surface, and wherein
the side wall is more hydrophobic than a surface of the first portion of the channel adjacent the side wall.

73. The microfluidic device of claim 72, wherein the side wall is a first side wall, and wherein the recessed portion of the channel further comprises a second side wall, the bottom surface disposed between the first and second side walls, and wherein
the second side wall is more hydrophobic than a surface of the second portion of the channel adjacent the second side wall.

74. The microfluidic device of any one of claims 69-73, wherein the channel follows a non-linear path along the length of the channel.

75. The microfluidic device of claim 74, wherein the path is a serpentine path.

76. The microfluidic device of any one of claims 69-75, wherein
the channel is formed in one or more intermediate film layers.

77. A microfluidic device, comprising:
a substrate;
a first intermediate film layer disposed on the substrate, wherein a recessed channel is formed in the first intermediate film layer; and
a second intermediate film layer disposed on the first intermediate film layer, wherein a primary channel is formed in the second intermediate film layer, and wherein a first portion of the primary channel is disposed on a first side of the recessed channel and a second portion of the primary channel is disposed on a second side of the recessed channel opposite from the first side.

78. The microfluidic device of claim 77, wherein at least side surfaces of the recessed channel are more hydrophobic than adjacent surfaces of the primary channel.

79. The microfluidic device of claim 77, further comprising:
a top layer disposed on the second intermediate film layer, the top layer forming a top surface of the primary channel opposite the substrate.

80. The microfluidic device of claim 79, wherein the substrate, second intermediate film layer, and the top layer are bonded together with an adhesive.

81. The microfluidic device of claim 79, wherein the substrate, second intermediate film layer, and the top layer are bonded together without an adhesive.

82. The microfluidic device of claim 77, wherein the first intermediate film layer comprises a polymer, and a top surface of the first intermediate film layer comprises a hydrophilic coating.

83. The microfluidic device of claim 78, wherein at least the side surfaces of the recessed channel are more hydrophobic than an adjacent top surface of the first intermediate film layer exposed to the primary channel.

84. The microfluidic device of any one of claims 77-83, wherein the substrate comprises a glass.

85. The microfluidic device of any one of claims 77-84, wherein the substrate, the first and second intermediate film layers, and/or the top layer each has a thickness greater than or equal to 20 micrometers and less than or equal to 1 millimeter.

86. The microfluidic device of any one of claims 77-85, wherein the first and second intermediate film layers each comprises a plurality of intermediate film layers.

87. A method of operating a microfluidic device, the method comprising:
flowing a first liquid through a recessed channel disposed between a first portion of a primary channel and a second portion of a primary channel; and
mixing a first substance in the first portion of the primary channel with a second substance in the second portion of the primary channel after the first liquid is flowed through the recessed channel.

88. The method of claim 87, wherein the first substance comprises a second liquid.

89. The method of claim 88, wherein the second substance comprises a third liquid.

90. The method of claim 89, further comprising:
flowing the second liquid through the first portion of the primary side channel; and
flowing the third liquid through the second portion of the primary side channel.

91. The method of any one of claims 87-88, wherein the second substance comprises a solid.

92. The method of claim 91, wherein mixing the first substance with the second substance reconstitutes the solid.

93. The method of any one of claims 87-92, wherein the first portion of the primary channel is isolated from the second portion of the primary channel prior to the first liquid flowing through the recessed channel.

94. A microfluidic device comprising:
a first layer;
a second layer disposed on the first layer; and
a third layer disposed on to the second layer, wherein a portion of the third layer is directly disposed on the first layer, and wherein the portion of the third layer forms at least a portion of a microfluidic channel.

95. The microfluidic device of claim 94, wherein:
the third layer is disposed on the first layer at a first location,
the third layer is disposed on the second layer at a second location, and
the third layer includes an inclined portion between the first and second locations.

96. The microfluidic device of claim 95, wherein the inclined portion is angled relative to a surface of the third layer at the second location.

97. The microfluidic device of claim 96, wherein the inclined portion is angled relative to the surface of the third layer at an angle greater than 0 degrees and less than 89 degrees.

98. The microfluidic device of claim 94, wherein the microfluidic channel extends in a first direction.

99. The microfluidic device of claim 98, wherein the inclined portion is angled at least partially in the first direction.

100. The microfluidic device of claim 98, wherein the inclined portion is angled at least partially in a second direction transverse to the first direction.

101. The microfluidic device of claim 94, wherein the first layer is a substrate layer.

102. The microfluidic device of claim 94, wherein the portion of the third layer is adhered to the first layer.

103. The microfluidic device of claim 94, wherein the third layer is a film.

104. A microfluidic device comprising:
a microfluidic channel extending in a first direction, the microfluidic channel comprising a first portion and a second portion, wherein the second portion is spaced from the first portion in a second direction transverse to the first direction, wherein a surface of the microfluidic channel includes an inclined portion between the first and second portions, wherein a height of the first portion is less than a height of the second portion, and wherein the heights of the first and second portions extend in a third direction perpendicular to both the first and second directions.

105. The microfluidic device of claim 104, further comprising a reagent disposed in the second portion of the microfluidic channel.

106. The microfluidic device of claim 105, wherein the reagent is disposed along the second portion of the microfluidic channel in the first direction.

107. The microfluidic device of claim 104, wherein the microfluidic channel comprises an inlet and an outlet, wherein the outlet is spaced from the inlet in the first direction.

108. The microfluidic device of claim 107, wherein when fluid is introduced into the inlet of the microfluidic channel, the fluid flows at least partially in the second direction before reaching the outlet.

109. A method comprising:
introducing fluid into a first portion of a microfluidic channel, the microfluidic channel extending in a first direction;
flowing the fluid in a second direction transverse to the first direction from the first portion of the microfluidic channel to a second portion of the microfluidic channel, wherein the first portion of the microfluidic channel and the second portion of the microfluidic channel are at least partially coextensive along a length of the microfluidic channel; and
flowing the fluid out of the second portion of the microfluidic channel to an outlet of the microfluidic channel, the outlet spaced from the first portion in the first direction.

110. The method of claim 109, further comprising mixing the fluid with a reagent disposed in the microfluidic channel prior to flowing the fluid out of the second portion of the microfluidic channel.

111. The method of claim 110, wherein mixing the fluid with the reagent comprises reconstituting a dried reagent.

112. The method of claim 109, wherein flowing the fluid in the second direction comprises flowing the fluid across an inclined surface.

113. The method of claim 112, wherein flowing the fluid in the second direction comprises flowing the fluid along a capillary pressure gradient.

114. The method of claim 109, wherein flowing the fluid in the second direction comprises flowing the fluid along a capillary pressure gradient.
